(19) United States
(12) Patent Application Publication
(10) Pub. No.: US 2018/0074074 A1
(43) Pub. Date: Mar. 15, 2018

(54) HOMOGENOUS AND HETEROGENEOUS ASSAYS AND SYSTEMS FOR DETERMINATION OF OCULAR BIOMARKERS

(71) Applicant: INSIGHT INSTRUMENTS, INC., Stuart, FL (US)

(72) Inventors: Klaus Peter LULOH, Stuart, FL (US); Ross MCDONOUGH, Stuart, FL (US); Michael ANNEN, Ft. Pierce, FL (US); Jade GOLD, Stuart, FL (US)

(21) Appl. No.: 15/437,351

(22) Filed: Feb. 20, 2017

Related U.S. Application Data
(60) Provisional application No. 62/393,996, filed on Sep. 13, 2016.

(51) Int. Cl. G01N 33/68; B01L 3/00; G01N 33/53

(52) U.S. Cl. CPC G01N 33/6893; G01N 33/6869; B01L 3/502; G01N 33/5302; G01N 2333/475; G01N 2333/5412; A61B 10/0045; B01L 2200/04; B01L 2300/123; B01L 2300/044; B01L 2300/042; B01L 2300/0672; G01N 2800/16; B01L 2400/06

(57) ABSTRACT

Disclosed herein are systems and methods for easy and rapid detection of ocular analytes in vitreous humor or aqueous humor. Specifically, exemplified are systems having a sample acquisition device that is in line with an analyte detection device. The system embodiments allow for the easy procurement and testing of samples. In a typical embodiment, the analyte detection assay device includes a housing that has a reaction chamber that is in fluid communication with a sample conduit and which contains reagents that specifically interact with the analyte. The reaction chamber is in fluid communication with a shunt that is in fluid communication with the sample conduit. The sample conduit has a sample conduit valve that is positioned distally to the shunt.

### BACKGROUND

### Field of the Invention

The invention relates to the general field of assay systems, devices and methods for detection of an analyte. Preferred embodiments of the invention preferably relate to detection of biomarkers for ophthalmic diseases such as vitreoretinal diseases and conditions of the eye in a subject in a sample taken from the subject, for example a vitreous humor sample.

### Description of the Background

Vitreoretinal diseases, such as AMD, CRVO, and PDR, are leading causes of blindness and vision loss worldwide. The retina is an extension of the brain that lines the eye interior. It contains the light sensitive nerve endings (photoreceptors, comprised of rods and cones) that allow vision. The vitreous humor (also referred to as the vitreous or the vitreous body) is a clear, gelatinous substance that fills the eye behind the lens, contacts the retina, and helps to keep the retina in place. In vitreoretinal disease states, biomarkers such as the cytokines VEGF (vascular endothelial growth factor), IL-6 (interleukin 6), or MCP-1 (monocyte chemotactic protein 1) have been documented to exist in elevated levels in the vitreous. The presence and amount of such biomarkers in the vitreous can be used to diagnose and to determine the stage or severity various vitreoretinal diseases, including, but not limited to, AMD, RVO, PDR, and trauma.

Vascular endothelial growth factor (VEGF) is a signaling molecule that stimulates angiogenesis and can be detected in a number of disease states, including cancer and vitreoretinal diseases like AMD and PDR. IL-6 is a pro-inflammatory cytokine that stimulates an immune response after trauma, infection, cancer, and the like, and also can be detected in vitreoretinal disease. MCP-1 (also sometimes known as small inducible cytokine A2 or chemokine (C-C motif) ligand 2) is a cytokine in the CC chemokine family that recruits immune cells to the sites of inflammation and is produced by tissue injury or infection, for example of tissues in the central nervous system, and also can be detected in elevated levels in vitreoretinal diseases.

Antibody-based drugs such as Avastin™, Lucentis™, and Eyelea™ have demonstrated promising clinical results in the treatment vitreoretinal disease. However, the treatment regimens with these anti-VEGF or anti cytokine drugs consist of intravitreal injections at fixed (frequently monthly) intervals for sometimes unlimited duration at great financial cost to the patient, as such exacting considerable adverse effects on the patient's quality of life. Being able to readily measure and monitor the levels of cytokine biomarkers such as VEGF therefore affords great benefit to patients suspected of suffering from vitreoretinal disease or being treated for vitreoretinal disease, as it allows for a more accurate diagnosis of the disease state and a better determination of the effectiveness or potential effectiveness of treatment during the clinical time course.

There currently exits an unmet need in the art for methods for determining the presence and levels of biomarkers, especially cytokines, in many diseases and conditions, including vitreoretinal diseases and conditions of the eye. The present invention provides useful embodiments for aspects of such determination, including sample collection and testing, as well as methods useful in assisting appropriate treatment of subjects suffering from these diseases and conditions.

### BRIEF DESCRIPTION OF THE DRAWINGS

Embodiments of the present invention are illustrated by way of example, and not by way of limitation, in the figures of the accompanying drawings below.

FIG. 1 shows a diagram of a sample acquisition and analyte detection system embodiment.

FIG. 2 shows an alternative an analyte detection device embodiment.

FIG. 3 shows a diagram of a cartridge embodiment useful for detecting analyte(s) in a sample.

FIG. 4 shows a diagram of a cartridge embodiment useful for detecting analyte(s) in a sample.

FIG. 5 shows the cartridge embodiment of FIG. 4 that is overfilled.

FIG. 6 shows a diagram of a sample acquisition and analyte detection system embodiment.

FIG. 7 shows a zoom-in cross-section view in of a component of the system shown in FIG. 6.

FIG. 8 shows a zoom-in cross-section view in of an alternative component of the system shown in FIG. 6.

FIG. 9 shows a zoom-in cross-section view in of an alternative component of the system shown in FIG. 6.

### DETAILED DESCRIPTION

### 1. Introduction

Embodiments of the invention relate to the quick and accurate determination of certain biomarkers in fluid, gel, or tissue samples from a subject, preferably samples taken from or associated with the eye. Rapid clinical monitoring of important biomarkers of disease, for example inflammatory or pro-angiogenic factors in patients with vitreoretinal disease, is useful to prevent unnecessary repeat treatments (such as intravitreal injections) for patients and to reduce cost for both patients and health care providers.

Accordingly, embodiments of the present invention provide assays, systems, devices and methods for determining the presence of vitreoretinal biomarkers, in qualitative, semi-quantitative and quantitative form, in a biological sample. These assays, systems, devices and methods also can be used in general form to test for these biomarkers and other biomarkers in any fluid or gel sample taken from a subject, but preferably test for biomarkers of vitreoretinal disease in a sample taken from the eye of a subject, for example aqueous humor, vitreous humor, eye tissue, and the like, or a combination thereof.

By using embodiments of the invention, unnecessary treatment rates and unnecessary repeat intravitreal injections can be avoided, reducing costs and complications (for example due to injection as well as systemic toxicity), thus increasing quality of life for the patient. A specific embodiment of a system includes sample collection device, such as a portable vitrectomy system with a sharp-tipped disposable probe, and an in-line analyte detection device for detecting analyte in the sample.

### 2. Definitions

The following terms as used herein have the following definitions. Unless otherwise defined, all technical and scientific terms used herein are intended to have the same meaning as commonly understood in the art to which this invention pertains and at the time of its filing. Although various methods and materials similar or equivalent to those described herein can be used in the practice or testing of the present invention, suitable methods and materials are described below. However, the skilled should understand that the methods and materials used and described are examples and may not be the only ones suitable for use in the invention. Moreover, because measurements are subject to inherent variability, any temperature, weight, volume, time interval, pH, salinity, molarity or molality, range, concentration and any other measurements, quantities or numerical expressions given herein are intended to be approximate and not exact or critical figures, unless expressly stated to the contrary. Hence, where appropriate to the invention and as understood by those of skill in the art, it is proper to describe the various aspects of the invention using approximate or relative terms and terms of degree commonly employed in patent applications, such as: so dimensioned, about, approximately, substantially, essentially, consisting essentially of, comprising, and effective amount. The terms front, back and side are only used as a frame of reference for describing components herein and are not to be limiting in any way.

The terms “first,” “second,” and the like, as used herein, do not denote any order, quantity, or importance, but rather are used to distinguish one element from another. The terms “a” and “an” do not denote a limitation of quantity, but rather denote the presence of at least one of the referenced item. The modifier “about” used in connection with a quantity is inclusive of the stated value and has the meaning dictated by the context. All ranges disclosed within this specification are inclusive and are independently combinable. Furthermore, to the extent that the terms “including,” “includes,” “having,” “has,” “with,” or variants thereof are used in either the detailed description and/or the claims, such terms are intended to be inclusive in a manner similar to the term “comprising.” The terms front, back and side are only used as a frame of reference for describing components herein and are not to be limiting in any way.

Unless otherwise defined, all technical and scientific terms used herein are intended to have the same meaning as commonly understood in the art to which this invention pertains and at the time of its filing. Although various methods and materials similar or equivalent to those described herein can be used in the practice or testing of the present invention, suitable methods and materials are described below. However, the skilled should understand that the methods and materials used and described are examples and may not be the only ones suitable for use in the invention. Moreover, it should also be understood that as measurements are subject to inherent variability, any temperature, weight, volume, time interval, pH, salinity, molarity or molality, range, concentration and any other measurements, quantities or numerical expressions given herein are intended to be reasonable approximations and not exact or critical figures unless expressly stated to the contrary. Hence, where appropriate to the invention and as understood by those of skill in the art, it is proper to describe the various aspects of the invention using approximate or relative terms and terms of degree commonly employed in patent applications, such as: so dimensioned, about, approximately, substantially, essentially, consisting essentially of, comprising, and effective amount.

The term “analyte” refers to any compound or composition to be measured in an assay, for example a biomarker or a portion thereof. Such an analyte also is referred to as a target or target analyte, and is capable of binding specifically to a capture molecule, which can be an antigen, hapten, protein, drug, metabolite, nucleic acid, ligand, receptor, enzyme, aptamer, antibody or fragment thereof, affibody, affimer, avimer, aptamer, aptide, cell, or cytokine. Preferably, the analyte is VEGF or a portion thereof, such as an epitope or hapten thereof, or is IL-6 or MCP-1, however any biomarker can be tested and detected using these methods. Analytes also can include antibodies and receptors, including active fragments or fragments thereof. An analyte can include an analyte analogue, which is a derivative of an analyte, such as, for example, an analyte altered by chemical or biological methods, such as by the action of reactive chemicals, such as adulterants or enzymatic activity.

The term “ocular analyte” refers to an analyte directly or indirectly pertaining to a marker related to an eye disease or condition. An ocular analyte may include, but is not limited to, an angiogenic ocular analyte or an inflammatory ocular analyte. Specific examples of angiogenic ocular analytes include, but are not limited to, VEGF and isoforms thereof, C-kit Y703, c-kit Y719, MMP-2, MMP-9, retinol binding protein-4 (RBP4), Secreted Protein Acidic and Rich in Cysteine (SPARC), Akt., VEGFR, EGFR, Bcr-Abl, Her2-Neu (erbB2), TGFR, PDGR, PDGFR, FGF, FGF-R, and PEDF. Specific examples of inflammatory ocular analytes include, but are not limited to, BAD Ser112, Bcl-2, C-abl, CC9 D330, Fadd 5194, TNF-alpha, IL-1, IL-1B, IL-6, IL-6R, IL-8, IL-10, IP-10, and MCP-1. “Indirectly pertaining to a marker” refers to and includes scenarios where the analyte detected is known to correlate with a level of another marker or molecule of interest either in the sample or in the biological context. For example, high levels of mRNA encoding IL-6 in a certain tissue or fluid may be understood to correlate with a known level of IL-6 protein in the same tissue or fluid, thus detection of this mRNA analyte could provide useful information concerning the level of IL-6. In addition, a marker byproduct can be detected as the analyte as opposed to the marker molecule of interest.

The term “antibody” is used here in its broadest sense refers to an immunoglobulin, or derivative or fragment or active fragment thereof, having an area on the surface or in a cavity which specifically binds to and is thereby defined as complementary with a particular spatial and polar organization of another molecule. The antibody can be monoclonal or polyclonal and can be prepared by techniques that are well known in the art such as, for example, immunization of a host and collection of sera or hybrid cell line technology, or recombinant technology. The term includes monoclonal antibodies, polyclonal antibodies, anti-idiotypic antibodies, synthetic antibodies, multispecific antibodies (e.g., bispecific antibodies) formed from at least two intact antibodies, and antibody fragments so long as they exhibit the desired binding activity. The antibodies can be chimeric antibodies, including humanized antibodies as described in Jones et al., Nature 321:522-525, 1986, Riechmann et al., Nature 332:323-329, 1988, Presta, Curr. Opin. Struct. Biol. 2:593-596, 1992, Vaswani and Hamilton, Ann. Allergy, Asthma & Immunol. 1:105-115, 1998, Harris, Biochem. Soc. Transactions 23:1035-1038, 1995, and Hurle and Gross, Curr. Opin. Biotech. 5:428-433, 1994. Antibodies of any class or isotype (e.g., IgA, IgA1, IgA2, IgD, IgE, IgG, IgG1, IgG2, IgG3, IgG4, and IgM) can be used.

The term “antibody” also refers to any antibody fragment(s) that retain a functional antigen binding region. Examples of antibody fragments include Fab, Fab′, F(ab′)2, and Fv fragments; diabodies; linear antibodies; single-chain antibody molecules; and multispecific antibodies formed from antibody fragments, all of which are known in the art. Papain digestion of antibodies produces two identical antigen-binding fragments, called “Fab” fragments, each with a single antigen-binding site. Pepsin treatment yields an F(ab′)2 fragment that has two antigen-combining sites and is still capable of cross-linking antigen. Fab′ fragments differ from Fab fragments by the addition of a few residues at the carboxy terminus of the heavy chain CH1 domain including one or more cysteines from the antibody hinge region. F(ab′)2 antibody fragments are pairs of Fab′ fragments which have hinge cysteines between them. Other chemical couplings of antibody fragments are also known. “Fv” is the minimum antibody fragment which contains a complete antigen-binding site. Diabodies are described more fully in, for example, European Patent No. 404,097, International Patent Application WO 1993/01161, Hudson et al., Nat. Med. 9:129-134, 2003, and Hollinger et al., PNAS USA 90: 6444-6448, 1993. Triabodies and tetrabodies also are described in Hudson et al., Nat. Med. 9:129-134, 2003.

The term “antibody,” as used herein, also includes antibody substitutes or any natural, recombinant or synthetic molecule that specifically binds with high affinity and specificity to a particular target. Thus, the term “antibody” or the term “antibody substitute” includes such synthetic antibodies or antibody substitutes such as aptamers, affibodies, affimers, avimers, aptides, and the like. Therefore, when describing the assay systems, devices and methods according to embodiments of the invention here, use of the term “antibody” for use as, for example, a reagent in the assay, indicates any of these alternatives also can be used.

The term “aptamer” refers to a nucleic acid or peptide molecule that specifically binds to a molecule of interest (target) with high affinity and specificity. Generally, aptamers are engineered through repeated rounds of in vitro selection or equivalently, SELEX (systematic evolution of ligands by exponential enrichment) to bind to various molecular targets such as small molecules, proteins, nucleic acids, and even cells, tissues and organisms. The aptamer may be prepared by any known method, including synthetic, recombinant, and purification methods, and may be used alone or in combination with other aptamers specific for the same target.

The term “biomarker” refers to an analyte that appears or increases in amount as an indicator of or marker of a particular disease or condition. In particular, this term includes any naturally occurring and detectable biological molecule, the presence of which, or the presence of which at or above a certain concentration or amount, indicates a vitreoretinal disease. For example, VEGF-A is a signaling molecule that stimulates angiogenesis, which is present in a number of disease states, including vitreoretinal disease (e.g., AMD. PDR) and cancer. Other biomarkers contemplated as useful with the present invention include IL-6, IL-8, IP-10, monocyte chemoattractant protein (MCP-1), major intrinsic protein (MIP), macrophage inflammatory protein (MIPr), PDGF, TNF, and the like, and include the analytes discussed above as angiogenic ocular analytes or inflammatory ocular analytes. The term “biomarker” also can refer to any analyte used to determine the presence or degree of a vitreoretinal disease or condition.

The term “fluid communication” as used herein with respect to one or more components of a system means that fluid (gas, liquid, and/or semi-liquid) can be transferred directly or indirectly from one component to another. Direct fluid communication pertains to fluid transfer from a first component that is contiguously associated with a second component. Indirect fluid communication pertains to fluid transfer from one component to another that involves passage through an intervening component.

The term “immobilized” (with respect to capture reagents or other reagents described herein) means that the migration of the capture molecule on the membrane or other surface on which it is immobilized (e.g., due to capillary flow of fluid such as the sample) or its escape from its immobilized location on the membrane is substantially impeded and, in certain embodiments, completely impeded. Methods for immobilizing the capture reagent are known in the art.

The term “label,” as used herein, refers to a substance, compound or particle that can be detected, particularly by visual, colorimetric, fluorescent, radiation, physical, magnetic, or instrumental means, for example, any material that is recognizable or detectable at very low concentrations and can be attached to a molecule to be detected, a test reagent that can bind the target to be detected, and the like.

The term “light” as used herein is intended to include a device that can generate of electromagnetic radiation.

The term “operable contact” refers to direct or indirect contact (intervening component) between a first and second component of a test device whereby the contact allows fluid to flow from one component to another via gravity, capillary action or any other fluid flow.

The term “sample” refers to any acquired material to be tested for the presence or amount of an analyte. Preferably, a sample is a fluid sample, preferably a liquid or gel sample. Examples of liquid samples that may be tested using a test device of the present invention include bodily fluids including blood, embryonic fluid, serum, plasma, saliva, urine, ocular fluid, vitreous humor of the eye, aqueous humor of the eye, semen, and spinal fluid; preferably the sample is obtained from the eye. The term “sample” also includes material that has been collected from a subject and treated further, for example solubilized or diluted in a solvent suitable for testing.

The term “reagent” refers to a molecule that is used to detect a target analyte, including reagents that bind to the target (e.g. capture molecule), agents that bind to the target-binding reagent, detectably labeled reagents and the like. The reagent can be an antibody, an aptamer, an aptide, an avimer, an affibody, an affimer, or any specific binding partner known in the art that can bind the target with high affinity.

The terms “capture molecule” or “capture reagent” are used interchangeably herein to refer to an antibody or antibody substitute that specifically binds to the target analyte to be detected. The capture reagent may include a detectable label associated therewith.

The term “therapeutic agent” as used herein is a pharmacologic agent useful in treating a disease or condition, preferably an eye disease or condition. These can include both naturally occurring substances (in purified form, partially purified form, or in unpurified form). Naturally occurring substances includes proteins, nucleic acids, fatty acids, steroids and other organic compounds produced in plants, animals, microorganisms or from non-living sources. In another aspect, a therapeutic agent may be a non-naturally occurring substance including proteins, nucleic acids, fatty acids, steroids and other organic compounds. A non-naturally occurring therapeutic agent can include modified natural products or substances without naturally occurring homologues.

Examples of therapeutic agents include antibodies, receptor agonists and antagonists, signaling pathway agonists and antagonists, small molecules, proteins, nucleic acids and other active agents known in the art. A therapeutic agent can be from any class of drugs, such as anti-angiogenesis agents, cancer treatments, anti-inflammation, molecules involved in growth, anti-apoptosis agents, steroid compounds used for reduced swelling, and monoclonal antibodies and fragments thereof. In one aspect, inhibitors of TNFalpha or inhibitors molecules in the signaling pathways of TNF-R1 or TNF-R2 are therapeutic agents.

In another aspect, a therapeutic agent is an inhibitor of VEGF, such as bevacizumab (Avastin®), pegaptanib (Macugen®), ranibizumab (Lucentis®), aflibercept (Eyelea®), Dexamethasone (Decadron®) and triamcinolone (Aristocort®). Bevacizumab is a recombinant humanized mouse monoclonal antibody that binds to and inhibits vascular endothelial growth factor A (VEGF-A). Ranibizumab is a monoclonal antibody fragment (Fab) from the same monoclonal antibody as Avastin, and also binds to VEGF. Aflibercept is a recombinant fusion protein comprising portions of the extracellular domains of human VEGF receptors 1 and 2 fused to the Fc portion of human IgG1, and acts as a soluble decoy receptor that binds VEGF. All of these compositions inhibit the binding and activation of cognate VEGF receptors.

The present invention also provides for identifying a subject who would benefit from administration of a different therapeutic agent. In such an aspect, the therapeutic agent may be something other than the previously administered treatment, for example without limitation, not bevacizumab, not pegaptanib, not ranibizumab, not Dexamethasone, or not triamcinolone.

The term “vitreoretinal disease,” as used herein, refers to any disease of the retina and/or vitreous body of the eye. Such diseases include such conditions as age-related or idiopathic macular degeneration, retinal detachments or tears, retinopathy of prematurity, retinoblastoma, uveitis, cancer of the eye, retinitis pigmentosa, macular holes, macular edema, BRVO, CRVO, flashes and floaters, and diabetic retinopathy.

### 3. General Provisions

The invention is described herein with reference to specific embodiments thereof. Various modifications and changes, however, can be made to the invention without departing from the broader spirit and scope of the invention. The specification and drawings are, accordingly, illustrative rather than restrictive. Throughout this specification and the claims, unless the context requires otherwise, the word “comprise” and its cognates, such as “comprises” and “comprising,” imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements. Unless otherwise clear from the context, a numerical value presented herein has an implied precision given by the least significant digit. Thus a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5× to 2×, for example, about 100 implies a value in a range from 50 to 200. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

### 4. Detailed Description of Embodiments

### A. Introduction

Modern analytical assays that are able to specifically detect and/or quantitate a particular substance (the analyte) typically are based on the ability to produce a capture molecule that can specifically recognize and bind small amounts of the analyte in a complex biological sample. The most common of these types of assays, for example for detection of biologically or medically important substances in a patient sample, is the immunoassay, in which the capture molecule is an antibody raised against the analyte.

The basic components of analytical assays are the analyte, also referred to as the target molecule or target analyte, the capture molecule, often a specific antibody or antibody substitute, and a detectable label of some sort. In general, the sample containing the analyte to be detected is mixed with one or more capture reagent that specifically binds the analyte and allows the analyte to be detected and/or separated from the remainder of the sample. In some assays, the capture reagent is labeled in some manner, allowing for direct detection. In others, the capture reagent is detected indirectly.

Analytical assays come in many different formats, including multi-step separation assays and homogenous (non-separation) assays, and can be performed in solution (liquid phase) or solid phase. Preferably, analytical assays are suitable for use in the laboratory, home, clinic or outpatient facility, and the like, and are intended to give an analytical result which is rapid and which requires a minimum degree of skill and involvement from the user. The most preferred assays are designed to detect biomarkers indicative of a disease state or condition in samples taken from the eye of a subject.

### B. Assays

Assays according to the invention are biochemical tests that detect, in a specific and qualitative, semi-quantitative or quantitative manner, the presence of a target analyte in a sample. Specific binding assays of this type rely on the ability of a specific-binding “capture” molecule to bind to the target analyte to be detected or measured without appreciable binding to any other component of a complex sample containing numerous other macromolecules. Commonly, these types of tests are referred to as ligand binding tests, or, for example, immunoassays. A detection method is used to determine the presence and extent of the binding which occurs, therefore the assay involves a label or other means to produce a detectable or measurable signal in response to this binding. Many different labels or other mechanisms are available to permit detection of the signal through different means, such as detection of radiation, color change or intensity, fluorescence, chemiluminescence, enzyme activity, physical agglutination or clumping, and the like. Steps in a typical assay of this type usually involve (1) sample collection and preparation; (2) analyte capture; and (3) detection. Examples of assays that may be implemented to detect analytes useful in accord with the devices, systems and method embodiments herein are further described below.

Sample collection can be performed according to any of the methods known in the art for collecting a bodily fluid, cellular, tissue or other sample. Any sample which contains or is suspected of containing the target analyte to be detected in the assay can be used. Samples can be taken from any subject, including human and animal subjects such as companion animals, laboratory animals, or livestock. Suitable subjects include, but are not limited to humans, simians, mice, rats, rabbits, dogs, cats, horses, cattle, sheep, and the like.

Fluid (liquid, semi-liquid, gelatinous, and the like) samples commonly are be collected by aspiration using a needle or collection in a vessel or by swab. Samples suitable for this type of collection include, but are not limited to blood, urine, pus, wound fluid, amniotic fluid, stool, saliva, tears, aqueous humor of the eye, vitreous humor of the eye, breast milk, emesis, sweat, nasal secretions, mucous, sputum, lymph, tumors, and the like. Fluid samples optionally are treated prior to assay by, for example, mixing, filtration, dilution or serial dilution, or centrifugation (e.g., to remove cells, cellular debris, or other particulates) to produce a better or cleaner sample for assay. When the sample is a solid or semi-solid material, including but not limited to stool, biopsy or autopsy tissue samples, and the like, it optionally is treated by maceration or dissolution, for example. Specialized equipment is available for unconventional or specialized sample collection, such as collection of vitreous humor of the eye, and can be used according to methods known in the art.

Target analytes which can be detected using the devices, systems and methods according to embodiments of the invention include any molecule for which a specific binding capture molecule can be found or made. Important analytes include nucleic acids, proteins, peptides, pharmaceuticals, hormones, biomarkers of disease, and the like. Most preferably, the analyte is a molecule of biomedical importance to diagnosis or treatment of a patient. In preferred embodiments, the analyte is of diagnostic significance, for example a biomarker, the presence of which indicates a disease or condition in the subject from whom the sample was taken. In some embodiments, if the analyte is a nucleic acid, the analyte in the sample optionally can be amplified by known methods of molecular biology such as PCR (polymerase chain reaction), or RT-PCR, prior to assay to increase the sensitivity of the method.

Capture of the analyte can be performed in solution or on a substrate using any convenient capture molecule. Most commonly, antibodies, such as polyclonal or monoclonal antibodies, or binding fragments thereof are used as the capture molecule, however any convenient capture molecule is suitable for use with the invention as long as it binds to the analyte specifically and with high affinity and specificity. Preferably, the capture molecule is able to bind the analyte at nanomolar concentrations or less, more preferably at picomolar or attomolar concentrations. Antibody substitute capture molecules such as aptamers, aptides, affibodies, affimers, avimers, and the like can serve as capture molecules, as well as receptors, specific binding partners, ligands, and the like.

Assays according to embodiments of the invention can be configured to operate in any convenient format known in the art. For example, the assay can be competitive or non-competitive, or a sandwich assay, and can be performed in solution (liquid phase) or on any of several known substrates. Some immunoassays can be carried out simply by mixing the reagents and sample and making a physical measurement, including newer “mix-and-measure” assays, which do not require the separation of bound from free ligand, for example bead-based assays. Such assays are called homogenous assays or less frequently non-separation assays. Multi-step assays are often called separation assays or heterogeneous assays. Commonly used assay types include radioimmune assays (RIA), immunoradiometric assays (IRA), enzyme-linked immunosorbant assays (ELISA), agglutination assays, precipitation or sedimentation assays, laterial flow (immuno)assays (LFIA), or blotting assays such as dot blots, western blots, and the like, each using any of the known capture molecules and detection systems. The assays according to embodiments of the invention can be automated using high throughput automatic analyzer instruments or robotic methods.

Many assays are named for the detection system which they employ, for example radioimmunoassays use a radioactive label, magnetic immunoassays use a magnet for separation, fluorescent immunoassays use a fluorescent label, while ELISA tests use an enzyme-substrate reaction to develop a detectable color. Fluorescent resonance energy transfer (FRET) systems and proximity ligation assays are other examples of assays that are described based on the detection system. Any of these assay types are contemplated for use with embodiments of the invention. Further description of detection methods is found below. Liquid phase ligand binding assays that rely on specifically binding capture molecules also include nucleic acid hybridization assays, which typically use an intercalating fluorescent dye that emits fluorescence via secondary structure conversion, molecular beacon capture of specific nucleic acid sequences, or real-time RT-qPCR using a molecular beacon or flurorophore intercalating dye.

A very simple form of assay is the “mix-and-measure” type or homogenous assay, in which the reagents are mixed together and the signal read. Specific examples of such assays are described in, for example, Kreisig et al., Scientific Reports 4:5613, 2014; Miskolci et al., Meth. Mol. Biol. 1172:173-184, 2014; Wang et al., Biosensors and Bioelectronics 26(2):743-747, 2010; Luu et al., http://www.kiko-tech.co.jp/products/intellicyt/ique_screener/intellicyt_hybridoma.pdf; Edelhoch, H., Hayaishi, O., and Teply, L.: The Preparation and Properties of a Soluble Disphosphopyridine Nucleotide Cytochrome C Reductase, J Biol Chem 197, 97, 1952; Mahler, H., Sarkar, N., Vernon, L., and Alberty, R.: Studies on Diphosphopyridine Nucleotide-Cytochrome c Reductase II. Purification and Properties, J Biol Chem 199, 585, 1952; Stowell et al., Anal. Biochem. 15:58-64, 2016; Einhorn et al., EPMA J. 6:23. 2015. Other homogenous assays that may be implemented with the system and method embodiments described herein include:
1. Fluorescence Polarization Immunoassay (FPIA) Maragas, Toxins, 2009 1:196-207;
2. Enzyme Multiplied Immunoassay (EMIT), Zherdev et al., Analytica Chimica Acta, 1997 347:131-138;
3. Dynamic Light Scattering. Nanoparticles conjugated with a capture molecule will bind to analyte contained in the sample creating a particle-biomolecular complex. These complexes can be detected using dynamic light scattering. See U.S. Pat. Nos. 8,883,094 and 9,005,994 and Liu et al. J. Am. Chem. Soc. 2008, 130, 2780-2782; for examples of detecting analytes using dynamic light scattering and metal particles;
4. Homogenous Temperature and Substrate Resolved Chemiluminescence Multi-analyte Immunoassay, See Kang et al., Analyst, 2009, 134:2246-2252; and
5. AlphaLISA assay (Perkin-Elmer, Waltham, Mass.). Ullman, E. F. et al. Luminescent oxygen channeling assay (LOCI): sensitive, broadly applicable homogeneous immunoassay method. Clin. Chem. 42, 1518-1526 (1996). McGiven, J. A. et al. A new homogeneous assay for high throughput serological diagnosis of brucellosis in ruminants. J. Immunol. Methods. 337, 7-15 (2008). This assay uses two different beads (alpha donor bead and AlphaLISA acceptor bead) that when both are bound to analyte, the acceptor bead can emit light at a certain wavelength upon excitation.

Assays according to the invention can be used on a purely qualitative basis, but also can be used with a measure of the intensity of the signal indicating binding to produce a quantitative or semi-quantitative result. For example, hand-held point-or-care analytical devices can provide a quantitative result by using unique wavelengths of light for illumination and either complementary-symmetry metal-oxide-semiconductor (CMOS; complementary metal-oxide-semiconductor) or charge couple device (CCD) detection technology to produce a readable image of the result. Using image processing algorithms specifically designed for a particular test type and medium, intensity is correlated with analyte concentrations. Other non-optical techniques for reporting quantitative results in the lateral flow test form include magnetic immunoassay (MIA).

Liquid phase binding assays are performed in solution. Solid phase specific binding assays provide very sensitive detection of analytes in fluid samples. These assays incorporate a solid support to which a capture molecule (such as an antibody, antibody substitute, antigen, hapten, receptor, analyte, receptor, ligand, and the like, or any member of a specific binding pair) is attached. The support can be any convenient substrate, including but not limited to the inside surface of a reaction vessel, a plate, tube, well, dipstick, microfluidic conduit, particles or beads made of a material such as polystyrene, nylon, nitrocellulose, cellulose acetate, glass fibers, poly-vinylidene fluoride), gold, magnetic material, polysaccharide (e.g., agarose), and the like. The reaction site or substrate on which the capture molecules are immobilized also is chosen to provide characteristics for detection of light absorbance. For example, the reaction site may be functionalized glass, Si, Ge, GaAs, GaP, SiO₂, SiN₄, modified silicon, or any one of a wide variety of gels or polymers such as (poly)tetrafluoroethylene, (poly)vinylidenedifluoride, polystyrene, polycarbonate, polypropylene, or combinations thereof. In general, any suitable or appropriate material(s) can be used in accordance with the present invention.

Methods for immobilizing the capture molecule on these substrates depend on the identity of the substance to be immobilized and the surface. These methods are well known in the art and can be chosen and/or modified according to need by any person of skill in the art.

Detection of the binding of capture molecule to target analyte can be achieved by any of a large number of known methods. Any of these methods are contemplated for use with embodiments of the invention. Examples of labeling and detection methods include, but are not limited to, radioactive isotope, enzyme-substrate, colorimetric and visual, fluorescence, chemiluminescence, magnetic, molecular beacons, and the like.

In certain embodiments, the assay platform is configured for multiplex detection of more than one analyte. Such assays employ two or more capture molecules, each of which specifically binds an analyte, and two or more detection methods so that the binding of each analyte can be determined. In preferred assays of this type, the target analytes include an angiogenic ocular analyte and an inflammatory ocular analyte. The dual detection can be performed in a single container where all the reagents for both assays are mixed together, or in two separate containers or vessels.

In one embodiment, homogenous temperature and substrate resolved chemiluminescence multi-analyte immunoassay format can be implemented to detect one or more analytes in a sample. See Kang et al., Analyst, 2009, 134:2246-2252 for explanation of this assay format.

Traditional competitive (homogenous) assays involve a competition reaction in which the target analyte in the sample competes for binding to a specific binding capture molecule (such as an antibody or aptamer, for example) with a labeled analyte reagent. After binding, the amount of the labeled, unbound analyte is measured. The more analyte present in the sample, the less labelled analyte reagent is able to bind to the capture molecule, therefore the amount of labeled, unbound analyte is inversely proportional to the amount of analyte in the sample. In a competitive (heterogenous) assay, unlabeled target analyte from the sample competes for binding to the capture molecule with a labeled analyte reagent as described above, however the labeled unbound analyte reagent is separated or washed away and the remaining labeled bound analyte is measured. Any of these types of assays, or variations thereof as known in the art, are contemplated for use with embodiments of the invention.

Commonly, the capture molecule is immobilized on a membrane, a reaction vessel surface or on suspended beads such as agarose beads, and detection is achieved using a labeled secondary binding molecule, such as an antibody or aptamer, that specifically binds to the primary capture molecule or to another binding region on the target analyte. If the capture molecule is immobilized on beads, separation and detection can be achieved using flow cytometry, magnetic separation, and the like. In addition, binding of the capture molecule and target analyte can be detected in solution without immobilization on a substrate.

In a typical non-competitive assay, the target analyte binds to a specific capture molecule that is labeled. After separating the unbound labeled capture reagent, the bound material is measured. The intensity of the signal is directly proportional to the amount of unknown analyte in the original sample. Alternatively, the assay is performed in a “sandwich” format where the target analyte binds to the capture molecule (which usually is bound to a surface for ease of separation) and labeled secondary capture molecule also binds to the target analyte. The amount of labeled capture molecule on the surface is then measured. The label intensity is directly proportional to the concentration of the analyte because labelled antibody will not bind forming a “sandwich” if the analyte is not present in the unknown sample.

Sandwich format binding ligand affinity assays can be performed with different detection methods. Typically, these assays are performed as solid-phase assays, where the target analyte is “sandwiched” between an immobilized capture molecule and a labeled capture molecule, each capture molecule binding to a different, non-overlapping epitope or binding area of the analyte. Immobilization allows the user to remove unbound substances from the bound analyte prior to detection with the labeled capture molecule. The primary capture molecule can be immobilized on any surface, for example the surface of the testing vessel (e.g., a multiwell plate), beads, a dipstick, filters, or column resins. The capture molecules (primary and secondary (labelled)) can be selected individually from antibodies, antibody substitutes, receptors, aptamers, nucleic acids, or any specific binding molecule. Most commonly these assays use an enzyme detection system, but any detection system can be used. Further labels and detection systems are discussed below.

An exemplary sandwich-type assay can be performed using a biotinylated aptamer or antibody capture molecule, immobilized on a streptavidin plate or beads. Sample containing the target analyte is incubated in a buffered solution with the immobilized capture molecule and then is washed away, leaving bound target analyte. A secondary capture molecule, such as an antibody or antibody substitute, then is incubated in a buffered solution with the bound target. The sandwich complexes are detected directly, by detecting the label on the secondary capture molecule, or indirectly using a labeled antibody that binds to the secondary capture molecule. These assays are known in the art and can be modified as necessary by a person of skill, including determining optimum concentrations of the reagents, and the like.

Competitive assays can be designed on a number of platforms and using various detection methods, however a two-step assay is preferable when greater sensitivity is required or the available sample size is small. In a typical two-step assay, sample containing the target analyte is exposed to immobilized capture molecules that bind the analyte. The immobilized analyte, bound to the capture molecules, then is exposed to a solution containing conjugated (labeled) analyte at a high concentration. This conjugated analyte saturates any of the immobilized capture molecules which are not bound to target analyte from the sample. Before equilibrium is reached and the previously bound target analyte can be displaced, the conjugate solution is removed. The amount of label bound to the immobilized capture molecules is inversely proportional to the amount of analyte present in the sample.

“Pull-down assay” refers to an assay which comprises removal of a target from solution. This removal occurs when a capture molecule in solution or suspension is mixed with the sample containing the target analyte and specifically binds to it. The capture molecule is labeled or bound to a substrate which allows the bound material to precipitate, agglutinate or otherwise be physically separated, for example using simple gravity, a magnet, centrifugation, and the like. In an agglutination assay, capture molecules that are bi- or multimeric- (i.e., that possess two or more specific binding areas, like an antibody) or substrates bearing multiple capture molecules, bind to the target analyte, forming large complexes that clump, precipitate, or agglutinate in the solution and fall to the bottom of the testing vessel. These large complexes can be seen with the naked eye if large enough and contain a visible color, for example, or can be seen with the aid of a microscope. In some embodiments, the clumps also contain a label that can be detected by other means, or the clumped material can be analyzed by chromatographic means. Latex agglutination involves latex particles, preferably colored particles, which are coated with bound capture molecules, which form complexes in the presence of the target analyte. Pull-down assays are convenient methods to determine whether a physical interaction between the target analyte and the capture molecule has taken place, i.e., to determine the presence of the analyte or as a semi-quantitative assay to determine relative amounts of the analyte.

Lateral flow tests also known as lateral flow immunochromatographic assays, are simple devices intended to detect the presence (or absence) of a target analyte in sample (matrix) without the need for specialized and costly equipment. Typically, these tests are used for medical diagnostics either for home testing, point of care testing, or laboratory use. These tests are based on a series of capillary beds through and across which the sample fluid migrates from a sample area or sample pad, across defined areas that contain various reagents. A typical assay uses a conjugate pad, in which the conjugated capture molecule which binds specifically to the target analyte is located. Upon binding, the captured analyte continues to flow laterally to a second area where a secondary capture molecule binds and immobilizes the conjugate-analyte complex in a relatively small area. Once the complexes accumulate, the conjugate's label, usually a colored particle, becomes more concentrated and hence detectable, often by the accumulation of color. Lateral flow tests of this type can operate as either competitive or sandwich assays.

General background information regarding lateral flow immunoassay systems is provided in Lateral Flow Immunoassay, Raphael C. Wong and Harley Y. Tse (Editors), 2009, Humana Press, a part of Springer Science+Business Media, LLC. (Library of Congress Control Number 2008939893) and U.S. Pat. No. 8,011,228. A specific embodiment of a lateral flow test is as follows.

### C. Samples

Any part of a subject can be collected for testing according to embodiments of the invention. The sample can be cellular or acellular. Samples can be taken from organs such as brain, skin, eye, esophagus, mucous membrane, heart, lung, stomach, pancreas, liver, kidney, colorectal, gall bladder, urinary bladder, ureter, urethra, lymph node, spleen, breast, uterus, cervix, ovary, testicle, prostate, vascular, thyroid, endocrine gland, and the like. Tissues including but not limited to connective tissue (e.g., fibrous tissues, fat, cartilage, ligament, tendon, bone, bone marrow, blood), muscle tissue (e.g., cardiac muscle, striated muscle, smooth muscle), nervous tissue (e.g., brain, spinal cord, nerves, grey matter, white matter) or epithelial tissue (e.g., simple squamous, simple cuboidal, simple columnar, stratified squamous, stratified cuboidal, pseudostratified columnar, transitional, olfactory, respiratory, intestinal, germinal) are contemplated, including malignant, infected, damaged, or healthy tissues. Fluid samples also can be collected as samples for testing, including but not limited to blood, urine, saliva, semen, tears, cerebrospinal fluid, amniotic fluid, embryonic fluid, vaginal secretions, menstrual blood, pus, wound fluid, breast milk, emesis, sweat, stool, mucus, sputum, lymph, nasal secretions, stool, saliva, tears, ocular fluid, vitreous humor of the eye, aqueous humor of the eye, and the like.

These samples optionally are prepared for testing, for example by filtration, centrifugation and the like to remove cells, particles, clots, DNA or any unwanted material, or to solubilize a solid or semi-solid sample. For example, blood can be prepared to produce serum or plasma for testing. In addition, samples can be concentrated or diluted for testing, including serial dilutions.

The samples are collected in any convenient size, depending on the assay or assays to which it is to be subjected. The size of the sample is preferably one that allows for the least amount of potentially adverse effects on the patient but which allows for optimal accuracy in the detection of analytes. For example, the sample size may be between 1-500 μl. In a specific embodiment, the sample size is 10-100 μl. In an even more specific embodiment, the sample size is 25-65 μl.

### D. Sample Collection

Samples can be collected using any convenient device which is clean and preferably sterile. Collection cups, swabs, pipettes, aspirators, collection bags, catheters, syringes, scoops, needles, and the like are known in the art and are contemplated for use with embodiments of the invention.

Specialized collection devices also can be used, including a specialized tool for collection of semi-solid or gelatinous material such as the vitreous humor of the eye. Preferably, the sample acquisition or collection device has a cannulated needle with an aspiration inlet, and an aspiration conduit that is, removably or permanently, in fluid communication with the aspiration inlet and an aspirator. The cannulated needle in the sample acquisition device typically will include an elongated body having a tapered distal tip. The tapered distal tip preferably is sharp, to assist with entry into tissue. The aspiration inlet can be at the tapered distal tip, but typically is upstream so as to be positioned on the elongated needle body. In the context of obtaining eye fluid samples, particularly samples of vitreous humor or other viscous or gelled material, the acquisition or collection device also can include a cutting mechanism to assist with obtaining the sample. Examples of preferred such devices having a cutting mechanism include, but are not limited to, INTRECTOR® or RETRECTOR® (Insight Instruments™) systems, or those described in U.S. Pat. Nos. 5,487,725; 5,716,363; 5,989,262; 6,059,792; 7,549,972; 8,216,246; or 8,608,753. The aspiration inlet preferably receives fluid which flows into the aspiration conduit wherein a portion of the conduit resides within the elongated needle body and courses out of the acquisition device to another portion typically in the form of flexible tubing. The aspiration conduit can be directly or indirectly connected to the analyte detection device in a permanent or removable fashion. While some of the specific acquisition device embodiments disclosed herein are particularly adapted for acquisition of eye fluid samples, those skilled in the art will appreciate that acquisition devices designed for acquisition of other types of samples which are disclosed herein or are known in the art. In a specific embodiment, the sample collection and testing system is described in the Examples section below and related figures.

The sample collection unit typically allows a sample to be collected from a subject and delivered to the assay device where it can react with reagents contained within the assay device to produce signal indicating the presence of the analyte of interest. The sample collection unit may take a variety of configurations so long as it collects and delivers the sample of bodily fluid to the assay vessel or container. In some embodiments, the sample collection unit is in fluidic communication with one or more components of the assay device or assay vessel. The sample collection device can be configured to collect a sample from the subject and deliver a predetermined portion or amount of the sample to the assay device to be assayed. In this manner, the device automatically meters the appropriate volume of the sample that is to be assayed. The sample collection unit can comprise a sample collection well, a metering channel, and a metering element. Generally, the sample collection well collects the bodily fluid from the patient. The metering channel is in fluidic communication with the sample collection well and is dimensioned to collect the predetermined portion of the sample to be assayed. The metering element is adapted to prevent a volume of sample larger than the predetermined portion of the sample from being assayed.

In one embodiment, the aspirator of the collection device is a syringe or pump, which can be connected to the aspirator outlet of the collection device housing. In an alternative embodiment, the aspirator can be at least partially contained within the housing such that it may be actuated by the user thereby obviating the need for an aspiration outlet on the outside of the housing. In a further embodiment, the cannulated needle used in the sample collection device includes an elongated body having a tapered distal tip. Typically, the tapered distal tip is sharp to assist with entry into tissue. The aspiration inlet may be at the tapered distal tip, but is typically upstream so as to be positioned on the elongated needle body. In the context of obtaining eye fluid samples, particularly vitreous humor which is viscous, the acquisition device may include a cutting mechanism to assist with obtaining the sample.

The collection device optionally can be configured such that the collection device mates to, connects to or can be inserted into a portion of the assay device or the vessel in which the assay is to be performed, and which allows the collected sample to be delivered through a fluid conduit into the assay device. In one embodiment, the collection device can be operated to pierce a membrane, gasket, bladder, lid, or the like of the assay device so that the sample contained in the collection device can be delivered into the testing apparatus through the needle that has pierced the assay device. This delivery can be through a port, by pressure from a syringe, or in any convenient manner.

### E. Containers and Vessels

Another embodiment is a point-of-care system for detecting an analyte in a sample (e.g. vitreous humor) that includes a sample acquisition device. Preferably, the sample acquisition device has a cannulated needle with an aspiration inlet. The sample acquisition includes an aspiration conduit that is, removably or permanently in fluid communication with the aspiration inlet and an aspirator. The system further includes an analyte detection device that is in fluid communication with the aspiration conduit and the aspirator, whereby fluid acquired through the aspiration inlet is delivered to the analyte detection device for analysis.

The assay device can be any container, preferably a sealable container or vessel of sufficient volume to contain the assay reagents, any solvent which may be necessary, and the sample to be analyzed. The container can take the form of a cup with a lid that snaps or screws on to close the container, or of a tube such as a test tube or Eppendorf tube, a vial, a bottle, a cuvette, a lateral flow device, a titer plate or microtiter plate, or a single well of such plates, including any vessel or container which is known in the art or suitable to this purpose.

The devices of the present invention preferably function as handheld devices in a point-of-care system. The term “handheld” refers to a device that is both small and light enough to be easily held in an adult's hand, and can readily be placed by hand into operation within a point-of-care system. A handheld device of the present invention may assume a variety of overall configurations, such as rectangular, triangular, circular, oval, cylindrical, and so forth. Regardless of the overall configuration, a handheld device of the present invention typically is enclosed within rectangular dimensions of about 30×30×15 cm (length×width×height), or about 12×10×5 cm, or about 8×6×1.5 cm, and even smaller, such as about 7×5×1 cm.

A “point-of-care” system as used herein refers to a system that can be used at a patient's home, bedside, or other environment for performing any type of bodily fluid analysis or test outside of a central laboratory. A point-of-care system of the present invention can enable testing to be efficiently carried out by a patient or an assistant, a health care provider, and so forth. A point-of-care system preferably has dimensions and a configuration that allows it to be conveniently transported to a user's desired environment and readily used for testing.

In one embodiment, an analyte detection device for determining the presence or amount of a target analyte in a sample includes a reaction chamber that contains components such as diluent, buffer, preservatives, and the like, which aid in performing the assay, and reagents that specifically interact with the target analyte, directly or indirectly and form part of the assay reaction. In a specific embodiment, the reaction chamber includes reagents that facilitate detection of one or more analytes via a homogenous type assay, that does not require separation of the analyte from the sample or immobilization of a capture molecule or other reagents.

Alternatively, the reaction chamber can include a substrate for a solid phase assay. For example, the interior of the chamber can comprise a surface on which a capture molecule is bound or can be bound, or comprise beads, such as agarose beads, on which a capture molecule is bound. The reaction chamber also usually includes one or more capture molecules, which may be immobilized on a surface, dissolved in a solvent or present in a dry form. The reaction chamber also optionally contains a conjugate (typically labeled) reagent, and optionally additional reagents that specifically bind to the target analyte or to other capture molecules, and any of the reagents which would be required, depending on the configuration of the assay. In alternative embodiments, the assay device is configured to detect two or more different analytes in the sample and therefore contains the necessary reagents for such testing.

Any or all of the reagents can be in dry form or dissolved in a suitable solution. The reagents and other components optionally can be contained in a subchamber within the reaction chamber that can be pierced, punctured, broken, or otherwise physically compromised to release or introduce the reagents into the main reaction chamber or into a channel in fluidic communication with the reaction chamber for performance of the assay, by actuation by the user. In a preferred embodiment, a sample collection device contains a piercing needle or other sharp device to puncture or break open the reaction chamber or the subchamber when inserted therein, and can simultaneously release the one or more reagents in the subchamber into the main reaction chamber and deliver the sample into the reaction chamber for assay. In alternate embodiments, the reagents, such as the capture molecule (optionally immobilized on beads, particles or a dipstick) and conjugate molecule are contained in one or more separate containers and are transferred to the reaction chamber prior to performance of the assay. Reagents and other components may be contained in reactant chambers as fluids or dry reagents. In some embodiments there may be two, three, four, five, six, or more, or any number of reaction chambers or subchambers as are necessary to fulfill the purposes of the invention.

In some embodiments of the invention the assay device includes one or more waste chamber to trap or capture liquids or other reagents as they are used in the assay. Such a conformation allows multiple step assays to be performed by allowing reagents to be added to the main reaction chamber by compromising the integrity of a subchamber to release the contents, and then allowing the contents to enter a waste chamber after use so that another reagent or group of reagents can be released into the main reaction chamber. By this method, washing steps can be performed, or multiple reaction steps can be performed in sequence without retaining the prior solution and reagent. On-board waste chambers also allow the device to be easily disposable. In preferred embodiments, there is more than one waste chamber, however waste solutions can be evacuated from the assay device to the outside for disposal rather than being contained or stored in a waste chamber. The waste chamber or waste evacuation port preferably is in fluidic communication with the site or vessel/chamber where the reaction(s) of the assay occur. The assay device also can be in kit form, wherein the components are packaged separately, in groups, or in a unitary package.

### F. Targets

Any target for which a binding partner can be found or produced can be assayed using embodiments of the invention, including, but not limited to whole cells or parts of cells, bacteria, parasites, viruses, proteins, nucleic acids, organic molecules, oligo- and poly-saccharides, glycoproteins, lipids, lipoproteins, or any biological molecule of interest for medical or scientific reasons. For example, the assays can be used to detect antigens (such as tumor antigens, antigens found in autoimmune disease, and the like), antibodies (which indicate the presence of a specific pathogen or immune disease condition), pathogen proteins (e.g., bacterial, fungal, parasite, or virally-expressed proteins), hormones (e.g., thyroid hormones), proteins and peptides (e.g., interleukins, growth factors, peptide hormones or prohormones, gene products, enzymes and enzyme substrates, serum proteins, receptors, and the like), small molecules (e.g., pharmaceuticals, prodrugs, drug metabolites, vitamins, toxins, and the like), nucleic acids (e.g., DNA, mRNA, tRNA, rRNA, and the like, including allelic variations an mutations), biomarkers of disease (e.g., autoimmune antibodies, C-reactive protein, cancer markers, rheumatoid factors, VEGF, IL-6, MCP-1, IP-10 tissue markers, tumor markers, liver enzymes, and the like). Virtually any chemical or biological effector of any size can be a suitable target.

In preferred embodiments, the target analyte or analytes are ocular analytes. The term “ocular analyte” refers to an analyte directly or indirectly pertaining to a marker related to an eye disease or condition. An ocular analyte includes, but is not limited to, an angiogenic ocular analyte or an inflammatory ocular analyte. Specific examples of angiogenic ocular analytes include, but are not limited to, VEGF, C-kit Y703, c-kit Y719, MMP-2, MMP-9, retinol binding protein-4 (RBP4), Secreted Protein Acidic and Rich in Cysteine (SPARC), Akt., VEGFR, EGFR, Bcr-Abl, Her2-Neu (erbB2), TGFR, PDGR, PDGFR, FGF, FGF-R, and PEDF. Specific examples of inflammatory ocular analytes include, but are not limited to, BAD Ser112, Bcl-2, C-abl, CC9 D330, Fadd 5194, TNF-alpha, IL-1, IL-1B, IL-6, IL-6R, IL-8, IL-10, IP-10, and MCP-1. “Indirectly pertaining to a marker” refers to and includes scenarios where the analyte detected is known to correlate with a level of another marker or molecule of interest either in the sample or in the biological context. For example, high levels of mRNA encoding IL-6 in a certain tissue or fluid may be understood to correlate with a known level of IL-6 in the same tissue or fluid, thus detection of this mRNA analyte could provide useful information concerning the level of IL-6. In addition, a marker byproduct can be detected as the analyte as opposed to the marker molecule of interest.

A certain threshold level of analyte preferably is detected in a sample in order to provide beneficial information regarding the subject. Persons of skill are able to determine the optimal concentrations of the capture molecule, conjugate (labeled) molecule, and other reagents and assay components tor optimal sensitivity and accuracy.

Preferred targets are proteins or peptides, and more preferred targets are ocular analytes such as an angiogenic ocular analyte or an inflammatory ocular analyte. The most preferred target analytes include, but are not limited to, C-kit Y703, c-kit Y719, MMP-2, MMP-9, retinol binding protein-4 (RBP4), Secreted Protein Acidic and Rich in Cysteine (SPARC), Akt., VEGF, VEGFR, EGFR, Bcr-Abl, Her2-Neu (erbB2), TGFR, PDGR, PDGFR, FGF, FGF-R, PEDF, BAD Ser112, Bcl-2, C-abl, CC9 D330, Fadd 5194, TNF-alpha, IL-1, IL-1B, IL-6, IL-6R, IL-8, IL-10, IP-10, and MCP-1. VEGF, IL-6 and MCP-1 are the most preferred target analytes.

The assay device optionally contains a control reagent or a set of control reagents to indicate that the test has been completed satisfactorily. In general, the control involves a control capture molecule that binds to the conjugate or labeled molecule or to a control analyte.

### G. Capture Molecules

The choice of suitable reactants in an assay depends on the particular analytes being examined, the samples collected, the concentration and amount of the target analyte, and many other factors related to convenience. In general, any reactants capable of together, as a system, reacting with the target analyte either directly or indirectly to generate a detectable product, are suited for use in embodiments of the invention.

Any molecule that can specifically bind to the target analyte or an epitope, hapten or other portion thereof is contemplated for use with the invention as a capture molecule to bind the target for detection. The assays take advantage of a specific binding partner to the target analyte to capture the target to the virtual exclusion of other molecules in the sample. Most assays employ an antibody, preferably a monoclonal antibody or binding fragment thereof. Any specific binding partner can serve as the capture molecule, however. Preferably, the capture molecule specifically binds to the target analyte with high affinity, that is with a dissociation constant (Kd) of at least about 10⁻⁷M, preferably about 10⁻⁹ to about 10⁻¹³ M, more preferably about 10⁻¹⁰ to about 10⁻¹² M.

Specifically, the following non-limiting list of capture molecules are contemplated as suitable for use with the inventive systems, devices, assays and methods: polyclonal antibodies or antigen-binding fragments thereof, monoclonal antibodies or antigen-binding fragments thereof, aptamers, affibodies, affimers, avimers, aptides, peptides, oligomer nucleic acids, and the like, so long as the capture molecule is specific to the analyte of interest and is able to bind thereto specifically and with high affinity.

When the capture molecule or molecules are antibodies, monoclonal antibodies or a binding fragment thereof are preferred. Antibodies for use with the invention as capture molecules include antibodies raised to the target antigen or a portion or hapten thereof, or containing an idiotype that was so raised. The antibody or fragment can be monomeric, bimeric or multimeric, monospecific, bispecific or multspecific, and can include natural or synthetic antibodies, anti-idiotypic, chimeric or humanized antibodies. Antibody fragments include, but are not limited to Fab fragments, Fd fragments, Fv fragments, dAb fragments, F(ab′)₂ fragments, single chain Fv fragments, and the like. Diabodies, linear antibodies, single-chain antibody molecules. All of these antibody types are well known in the art and within the person of skill's ability to produce, select, and use.

Monoclonal antibodies can be produced, for example, according to any known method in the art, including the traditional hybridoma methods of Kohler and Milstein, Nature, 256:495, 1975 or recombinant methods according to Cabilly et al., U.S. Pat. No. 4,816,567 or Mage and Lamoyi, Monoclonal Antibody Production Techniques and Applications, pages 79-97. Marcel Dekker Inc., New York, 1987. The antibodies can be produced in any animal, but preferably are produced in a convenient mammal or are recombinant. Any mammal is suitable, including humans, simians, rats, mice, rabbits, dogs, cats, horses, cattle, sheep, and the like.

Antibodies specific against the ocular analytes useful in the embodiments discussed herein are known in the art and commercially available. Companies that supply such antibodies include Abcam™ (Cambridge, Mass.), Santa Cruz Biotech™ (Dallas, Tex.), Sigma Aldrich™ (St. Louis, Mo.), Cell Signaling Technology™ (Danvers, Mass.), R&D Systems™ (Minneapolis, Minn.), Novus Biologicals™ (Littleton, Colo.) and Life Technologies™ (Carlsbad, Calif.), inter alia. In certain embodiments, VEGF binding antibodies may include, but are not limited to, VEGF M1 or M2, Avastin™ (Genentech™, San Francisco, Calif.)), EYLEA™ (Regeneron™, Tarrytown N.Y.), and Lucentis™ (Genentech™, Inc.). VEGF coating antibodies may include VEGF P1, P2 or P3, VEGF RaH RD System™ or VEGF GaH PoeroTech™. An example of an anti-MCP-1 antibody includes ab9669 (Abcam™). An example of a SPARC antibody includes ab61383 (Abcam™). U.S. Patent publication US2010/0150920 and WO/2008/156752 are cited for information concerning ocular analytes and antibodies that can be used for detection. Secondary antibodies specific against IL-6 include, but are not limited to, monoclonal anti-human IL-6 antibody or alternatively, polyclonal anti-human IL-6 antibody (e.g. goat-anti-human IL-6, rat-anti-human IL-6, or rabbit-anti-human IL-6). An example of an anti-IL6 antibody includes ab6672 (Abcam™).

An aptamer is a small single-stranded nucleic acid (DNA or RNA) in a hairpin-loop, pseudoknot, G-quartet, or stem loop, configuration or a short variable peptide domain, attached at both ends to a protein scaffold to form a loop. These structures can be developed to specifically bind a particular three-dimensional structure similar to the way an antibody binds an antigen. Hence, aptamers and similar specific binding molecules sometimes are referred to as antibody substitutes. The aptamer binding structures are selected from a large random-sequence pool (or are found naturally in riboswitches) and fold into a well-defined three-dimensional structure that specifically binds with high affinity to a specific target molecule. Aptamers can be selected for specific binding to any molecular target, including proteins, peptides, and the specific biomarkers identified herein as preferred target antigens. Aptamers bind with high specificity and affinity, and can bind strongly. Upon recognition of their target, nucleic acid aptamers bond by internal complementary DNA/RNA base pairing. This base pairing creates secondary structures such as short helical arms and single stranded loops. See Tuerk and Gold, Science 249:505, 1990; Ellington and Szostak, Nature 346:818, 1990; Eaton, Curr. Opin. Chem. Biol. 1:10-16, 1997; Famulok, Curr. Opin. Struct. Biol. 9:324-9, 1999, and Hermann and Patel, Science 287:820-5, 2000 for further description of RNA and DNA based aptamers.

Nucleic acid aptamers typically are about 15-60 nucleotide bases long, but can be shorter or longer, including up to 200 nucleotides or more. They are generated using a combinatorial chemistry procedure termed “systematic evolution of ligands by exponential enrichment” (SELEX). The term “SELEX” refers to a combination of selecting nucleic acids that interact with a designated target molecule in the desired manner, usually by high affinity binding to the target, and amplification of those selected nucleic acids. This method identifies the nucleotide sequences (aptamers) that have the desired binding characteristics.

SELEX (a method for in vitro evolution of nucleic acids for the desired binding characteristics) involves preparing a large number of (usually randomized) candidate nucleic acids and binding a mixture of these candidates to the desired target, washing to remove unbound material, separating the bound nucleic acids, and isolating and identifying the bound sequences. These purified individual sequences are the aptamers. Usually, several rounds of selection and enrichment are performed to refine and improve the affinity of the selected aptamer, usually alternating with rounds of amplification of the sequences. Thus, starting with a randomized mixture, repeated cycles of contacting with the target under binding conditions, purifying bound sequences and amplifying the bound sequences, SELEX results in a ligand-enriched mixture of nucleic acids which can be repeated as many times as needed to yield a highly specific, strong-binding nucleic acid aptamer. This process is described in more detail in U.S. Pat. Nos. 5,475,096, 5,580,737, 5,567,588, 5,705,337, 5,707,796, 5,763,177, 6,011,577, and 6,699,843. Embodiments of the SELEX process in which the target is a peptide are described in U.S. Pat. No. 6,376,190, entitled “Modified SELEX Processes Without Purified Protein.” In the instant case, the targets include C. difficile toxin A, toxin B, binary toxin, binary toxin A chain, or binary toxin B chain. Another screening method to identify aptamers is described in U.S. Pat. No. 5,270,163. Any of the methods described in these patents can be used to produce aptamers suitable for this invention.

This same SELEX process can be used to select aptamers that have improved characteristics, including, but not limited to higher affinity or avidity, improved stability and the like. In addition, the aptamers can be modified as described in U.S. Pat. Nos. 5,660,985 and 5,580,737, using SELEX or photoSELEX procedures. In particular, SELEX can be used to identify aptamers that have desirable off-rate characteristics. See U.S. Patent Publication Nos. 2009/0004667 and 2009/0098549, which provides methods for improving (slowing) the disassociation rates for selected aptamers.

RNA aptamers can form diverse complex secondary and tertiary structures that bind the target with the entire sequence. Production of RNA aptamers requires reverse transcription, in which RNA is converted into DNA during their synthesis by SELEX, a step not necessary for DNA aptamers. DNA aptamers also form complex secondary and tertiary structures that bind the target with the entire sequence, but the possible three-dimensional structures are somewhat less diverse than RNA aptamers.

Peptide aptamers are short peptide sequences, usually about ten to twenty amino acids in length, attached as a loop (at both ends) to a protein scaffold. The scaffold can be any protein which is sufficiently soluble and compact. The bacterial protein thioredoxin-A is commonly used, with the variable loop inserted within the reducing active site (a -Cys-Gly-Pro-Cys- loop) in which the two cysteine residues can form a disulfide bridge.

Because peptide aptamers are small, simple peptides with a single variable loop region tied to a protein at both ends, the peptide aptamer tertiary structures are constrained by the protein scaffold to which they are attached, reducing flexibility and often therefore effectiveness. This structural constraint also, however, can greatly increase the binding affinity of a peptide aptamer to levels comparable to an antibody's (nanomolar) range.

Peptide aptamers that bind with high affinity and specificity to a target protein can be isolated by a variety of techniques known in the art. Peptide aptamers can be isolated from random peptide libraries by yeast two-hybrid screens as described in Xu et al., Proc. Natl. Acad. Sci. 94:12,473-12,478, 1997 or by ribosome display as described in Hanes et al., Proc. Natl. Acad. Sci. 94:4937-4942, 1997. They also can be isolated using biopanning and surface display technology, for example from combinatorial phage display libraries, mRNA display, ribosome display, bacterial display, yeast display, or chemically generated peptide libraries. See Hoogenboom et al., Immunotechnology 4:1-20, 1998. For example, small peptides can be displayed on a scaffold protein (e.g., one based on the FKBP-rapamycin-FRB structure and selected based on interactions between the peptides and the desired target molecule, controlled by the small molecule, rapamycin, or by non-immunosuppressive analogs. This process is known as “selection of ligand regulated peptide aptamers (LiRPAs).

Aptamer-based tests have several potential advantages over using antibodies: aptamers generally have lower molecular weight, provide higher multiplexing capabilities (low cross-reactivity, universally-applicable assay conditions), chemical stability (to heat, drying, and solvents, reversible renaturation), provide ease of reagent manufacturing, amenability to defined chemical modification, and consistent lot-to-lot performance, and can be produced at lower cost. Aptamers can be generated against virtually any protein target, including targets for which antibodies are not available or are difficult to produce; the wider range of possible targets for aptamers is due to their ability to structurally conform to the binding site on their targets and their selection without the need to produce an immune response. Further increasing the binding repertoire is the possibility to use non-natural bases, in lieu of the four natural bases.

Unlike nucleic acid aptamers, antibodies can produce undesirable immune responses. In addition, aptamers are more stable chemically, cheaper, and easier to produce than antibodies, are more consistent lot-to-lot and require less specialized equipment. DNA and RNA aptamers also can differ in sequence and folding pattern even when selected for the same target. Aptamers, however can be limited because sometimes the non-covalent bonds they form with target molecules can be too weak to be effective (i.e., they have a weak or fast off-time); these kinetic limitations may form the basis as why, to date, aptamers have not found utility in LFIA's. In addition, aptamers are digested by enzymes unless modified. Detection methods used in antibody-based tests also can be used in aptamer-based tests.

Aptamers can be modified, for example by combination with a ribozyme to self-cleave in the presence of their target molecule. Additional possible modifications include replacing the 2′ position of nucleotides with a fluoroamino or O-methyl group for enhanced nuclease resistance. A second addition in the form of a “mini hairpin DNA” can impart a more compact and stable structure that resists enzymatic digestion and extends the life of the aptamer in solution. Bridging phosphorothioates also can be added, as well as end caps to reverse polarity of the chain and linker sequences (e.g., PEG) for ease in conjugation. Adding an unnatural or modified base to a standard aptamer can increase its ability to bind to target molecules as well. Further, “secondary aptamers” also are contemplated for use with the invention in certain embodiments. Secondary aptamers are designed to contain a consensus sequence derived from comparing two or more known aptamers to a given target.

Avimers are artificial antibody mimetic proteins which can bind certain antigens by multiple binding sites. Avimers are made up of two or more peptides of about 30-25 amino acids each, connected by a linker. The peptides are derived from receptors for the target protein, usually the A domain of a membrane receptor, each binding to different epitopes on the same target. The multiple binding domains increases avidity for the target protein. Avimers also can be constructed with binding domains directed against epitopes on different targets, creating a bispecific antibody mimetic. Avimers generally bind to target in sub-nanomolar ranges, and are more stable chemically than antibodies. They can be produced by selecting for binding domains as is done for peptide aptamers, for example using display techniques such as phage display and panning in cycles.

Affimer molecules are small (about 12-14 kDa), highly stable recombinant proteins that mimic monoclonal antibodies by specifically binding to a selected target with two (or more) binding domains. The affimer protein is derived from the cysteine protease inhibitor family of cystatins and based on the cystatin protein fold, but can be modified with different tags and fusion proteins. Affimers contain two peptide loops and an N-terminal sequence that can be randomized and screened to discover sequences that strongly and specifically bind to a desired target, similarly to monoclonal antibodies. The peptides are stabilized by a protein scaffold that constrains the tertiary structure and thereby increases binding affinity. Affimers are stable to temperature and pH extremes, freezing and thawing, and generally have low steric hindrance compared to antibodies.

Affimers are easy to express at high yields using bacterial, mammalian, insect or any convenient cells. General methods are as follows: A phage display library of about 10¹⁰ randomized potential target-binding sequences is generated and screened to identify a sequence with the desired high affinity and specific binding. Multiple rounds of screening, purification and identification improve the characteristics of the identified molecule, and the protein sequence is generated using recombinant systems as known in the art.

Affibodies are small (generally about 6 kDa) engineered antibody-mimetic proteins originally based on the Z domain of protein A, which binds IgG. Currently, however, the scaffold for the binding site has been modified and substituted to create different surfaces. These protein scaffold molecules usually have a three alpha helix bundle structure. The binding site contains 13 randomized amino acid residues which are screened for binding to the desired target using phage display or other display technologies as described above. The affibody molecules then are expressed in a host cell, such as bacterial, mammalian or insect cells or are produced by chemical synthesis. Affibodies sometimes are fused head-to-tail to create bi- or multi-specific binding proteins. Affibodies can be produced with sub nanomolar or picomolar affinity for the target molecule, and are stable.

Aptides are high-affinity peptides described in United States Patent Publication No. 2011-0152500, where they are referred to as “bipodal-peptide binders” and in Kim et al., “Bio-inspired design and potential biomedical applications of a novel class of high-affinity peptides” Angew. Chem. Int. Ed. Engl. 51(8):1890-1894, 2012. Aptides are antibody substitutes with a stabilizing rigid linker or backbone and two short peptides which specifically bind a target. The peptides are randomized and selected for specific binding to a desired target as known in the art and described briefly herein. The linker can be a strand which forms a bound loop due to the presence of a parallel and an antiparallel strand, for example, or another structure which forms non-covalent bonds to hold the strands together to form a stable structure, preferably with a beta-hairpin motif. Aptides have been constructed with both L- and D-amino acids.

Specific binding partners such as receptor-ligand, enzyme-substrate, and any other known binding pairs with high specificity and affinity can be used to capture the appropriate target. These, or any, specific binding molecules are contemplated for use with the invention as reagents that bind to the target or as labeled detection reagents, in any portion of the herein described assay system and device, however antibodies or antibody substitutes such as aptamers, avimers, affimers, affibodies and aptides are preferred. The specific binding molecules can be used as a capture molecule, a substitute for a secondary antibody or binder, a labeled detection molecule, or any use in place of an antibody.

The capture molecules optionally are immobilized on a substrate. One skilled in the art will appreciate that there are many ways of immobilizing various reactants onto a support where reaction can take place. The immobilization may be covalent or noncovalent, via a linker moiety, or tethering them to an immobilized moiety. These methods are well known in the field of solid phase synthesis and micro-arrays (Beier et al., Nucleic Acids Res. 27:1970-1-977 (1999). Non-limiting exemplary binding moieties for attaching either nucleic acids or proteinaceous molecules such as antibodies to a solid support include streptavidin or avidin/biotin linkages, carbamate linkages, ester linkages, amide, thiolester, (N)-functionalized thiourea, functionalized maleimide, amino, disulfide, amide, hydrazone linkages, and among others. In addition, a silyl moiety can be attached to a nucleic acid directly to a substrate such as glass using methods known in the art. Any known method is contemplated for use with embodiments of the invention.

### H. Detection Methods and Materials

Any method known in the art for use in binding assays, such as immunoassays, can be used to detect the binding of target analyte to the capture molecule. Reagents suitable for detection of an analyte can be included in a reaction chamber of system embodiments described herein to allow for the rapid and straightforward detection, and quantification or semi-quantification, of analyte in the sample. A label that can produce a detectable signal can be attached to the capture molecule itself, to a second molecule that binds the target or that binds the capture molecule, for example, or any of a number of specialized labeling and detection methods can be used.

A “label” refers to one or more substances, compounds, complexes or particles that can be detected, preferably visually or instrumentally (such as colorimetric, fluorescent, radiographic, physical, and magnetic instrumentation), and preferably at low concentrations, such as less than 10⁻⁷ M, 10⁻⁸ M, 10⁻⁹ M, 10⁻¹⁰ M, 10⁻¹¹ M, 10⁻¹² M, or even less than 10⁻¹³ M. Labels include but are not limited to organic dyes (which can be detected by colorimetric instrumentation or visually), fluorescent markers (which can be detected by microscopy under excitation by specific light wavelengths), quantum dots, colored particles (which can be detected by visualization with the naked eye or by microscopy), nanoparticles, enzymes (which can be detected by exposure to an enzyme substrate which forms a detectable substrate in the presence of the enzyme), colloidal gold particles (which can be detected by electron microscopy or colorimetrically), and radioactive isotopes (which can be detected by scintillation counting or any other convenient method). Labels also can be bound to molecules known to assist in binding or in amplifying the detectable signal, such as biotin-streptavidin or biotin-avidin complexes, a tyramide signal amplification (TSA) in combination with Alexa Fluor™ dyes, chromogenic or chemiluminescent substrates, phycobiliproteins, fluorescent microspheres, and the like.

In specific embodiments, the label enables the detection of a target analyte via a homogenous assay. One example of a homogenous assay includes Förster resonance energy transfer (FRET). The principle of the homogenous FRET assay is based on a low-affinity labeled (donor) peptide or other molecule and a quenching (acceptor) molecule, both bound to a target analyte-specific capture molecule. When the donor and the acceptor moiety are in close proximity to each other fluorescent emission is reduced due to FRET. In FRET, the excitation light raises a “donor” fluor to an excited state, which results in the release of a photon. An “acceptor” molecule, which can be another fluor or a non-fluorescent molecule, is designed to be in close enough proximity with the donor fluor to absorb the emitted photon, which then effectively quenches the emitted light in the case of non-fluorescent molecules. The emitted photon also is quenched when using fluors as quenchers, and if the absorbed photon is within the excitation wavelength range of the second fluor, the acceptor electron will be raised to an excited state and release a photon at the wavelength of the acceptor fluor. The acceptor fluorophore emits light of a longer wavelength, which is detected in specific channels. The light source cannot excite the acceptor dye.

Fluorescence resonance energy transfer (FRET) probes can include a pair of fluorescent probes chosen so that the emission spectrum of one overlaps significantly with the excitation spectrum of the other. The typical FRET hybridization probe system consists of two oligonucleotides labeled with fluorescent dyes. The hybridization probe pair is designed to hybridize to adjacent regions on the target DNA. Each probe is labeled with a different marker dye. The donor probe is labeled with fluorophore at the 3′ end and the acceptor probe at 5′ end. Interaction of the two dyes can only occur when both are bound to their target. The donor fluorophore (F1) is excited by an external light source, then passes part of its excitation energy to the adjacent acceptor fluorophore (F2). The excited acceptor fluorophore (F2) emits light at a different wavelength which can then be detected and measured. A modification of the FRET pair would be a single bead conjugated to an antibody (or aptamer) which emits a chemical species in response to a specific wavelength of light; this chemical species would then activate a second antibody (or aptamer) conjugated bead which would then fluoresce. Only in the presence of analyte would the beads be in close enough proximity to have a reaction.

Contact quenching occurs when the fluorophore is complexed with a quenching molecule prior to excitation; because of direct contact of the fluorophore with the other molecule, the energy from excitation is immediately transferred to the contact molecule, and this energy is then lost by heat. Collision quenching occurs when an excited fluorophore reacts with a quencher molecule in solution, which immediately causes the transfer of energy to the contact molecule and the relaxation of the excited fluorophore.

Molecular beacons (also known as molecular beacon probes) are hairpin-shaped oligonucleotide hybridization probes with an internally quenched fluorophore that can report the presence of specific nucleic acids in homogenous solutions. By binding to specific DNA sequences, the fluorescence is restored when the hairpin structure is released and the fluorophore and quencher are no longer held in close proximity. These reagents are useful when it is not possible or desirable to isolate the probe-target hybrids from an excess of the hybridization probes.

A typical molecular beacon probe is about 25 nucleotides long, but can vary from about 10 to about 50 nucleotides. The center nucleotides are complementary to a nucleic acid target analyte, and do not base pair with one another, while the nucleotides at the termini are complementary to each other, forming the hairpin structure with a specifically binding loop. One of the termini contains a fluorophore and the other a quencher, so that when they are not bound to the target analyte, they are held in close proximity, quenching the fluorescence. A typical molecular beacon structure contains an 18-30 base pair loop region that hybridizes specifically to the target analyte, a stem formed by the hybridization of the termini of the loop (about 3 to 7 nucleotide residues on each terminus), a 5′ fluorophore at the 5′ end of the molecular beacon, and a 3′ quencher (non-fluorescent) dye covalently attached to the 3′ end of the molecular beacon. Binding of the loop region to the target changes the conformation of the molecular beacon, causing the fluorophore and quencher to move apart, removing the quench and producing fluorescence. Terminal sequences of shorter length are preferred so that the hybridization of the stem region is not too strong. This method can be used with aptamers in certain embodiments, in which case the loop region binds to the target analyte for which the aptamer was created, for example a protein.

The molecular beacon only hybridises to complementary nucleic acid sequences, which increases dramatically the ligand binding assay selectivity, but might reduce the assay sensitivity as well, because only one fluorophor is detected per binding event. However, molecular beacon detection can be used in conjunction with real-time quantitative PCR (RT-qPCR) can provide both selectivity and sensitivity, since the target analyte DNA is amplified. RT-qPCR can be used in conjunction with any assay where the analyte is a nucleic acid, to amplify the analyte, or where the label is a DNA probe

Color, fluorescence, luminescence, phosphorescence, radioactivity, magnetically and the like, as discussed in the art can be used. A label can be, for example, a pigment produced as a coloring agent or ink (such as Brilliant Blue, 3132 Fast Red 2R and 4230 Malachite Blue Lake), a particle (such as latex beads, colored latex beads, magnetic beads, carbon nanoparticles, particles of pigment, gold particles, a quantum dot, and nanoparticles of carbon, selenium or silver), an enzyme-substrate system (such as alkaline phosphatase and horseradish peroxidase), a radioactive label (such as ¹³¹I or ³H), a molecule that emits light (such as a phosphorescent, fluorescent, chemiluminescent or electrochemiluminescent molecule), and the like.

Various labels can be attached to capture molecules or other molecules via covalent attachment to charged amino acid side groups (i.e., ammonium or carboxylate groups), carbohydrate moieties, sulfhydryl groups and tyrosine residues if the molecule to be labeled is a protein. Nucleic acids also can be labeled by covalent attachment or by intercalating dyes. Radiolabeling can be achieved by substituting one or more atoms in the molecule with a different radioisotope, or by iodination of certain groups on the molecule. Proteins can be labeled by iodinating tyrosine residues, for example. In addition, biotin-avidin or biotin-streptavidin can be used to link labels to capture molecules, conjugate molecules or any other reagent. Guides to techniques useful for labeling antibodies and other proteins and for detection, have been published and are located at www.bionovuslifesciences.com.au/files/Innova/Guide_to_labeling_your_primary_antibody.pdf, www.thermofisher.com/us/en/home/life-science/protein-biology/protein-biology-learning-center/protein-biology-resource-library/pierce-protein-methods/overview-detection-probes.html, /www.thermofisher.com/us/en/home/life-science/protein-biology/protein-biology-learning-center/protein-biology-resource-library/pierce-protein-methods/fluorescent-probes.html, vectorlabs.com/data/brochures/MBB.pdf

Enzymes can be conjugated to proteins, for example, and to target or capture molecules for use in assays. These enzymes allow for detection often because they produce an observable color change in the presence of certain reagents. In some cases these enzymes are exposed to reagents which cause them to produce light or chemiluminescence. Horseradish peroxidase, alkaline phosphatase, glucose oxidase, beta-galactosidase, luciferase, and the like, or any suitable enzyme is conjugated to the substance to be labeled (for example using the periodate method or the Lightening-Link™ method), and is detected by exposure to a specific substrate of the enzyme that produces a colored reaction product. Horseradish peroxidase can be detected using a peroxidase chromogen such as Trinder reagents (TODB or TOOS (N-ethyl-N-(2-hydroxy-3-sulfopropyl)-3-methylaniline, sodium salt, dehydrate) used in combination with 4-aminoantipyrene, triaryl imidazoles, and ABTS (2,2′-azino-bis (3-ethylbenzthiazoline-6-sulfonic acid). This chemistry involves a reaction where two colorless organic molecules form a colored product in the presence of peroxidase and hydrogen peroxide, generating an intensely colored product. Other suitable substrates for use with enzyme detection systems include nitroblue tetrazolium with 5-bromo-4-chloro-3-indolyl phosphate, Fast Red TR/naphthol AS-MX with 4-chloro-2-methylbenzenediazonium/3-hydroxy-2-naphthoic acid 2,4-dinethylanalide phosphate, or pNPP substrates (alkaline phosphatase), molybdate-enhanced polyvinyl alcohol or starch-glucose-iodide (glucose oxidase), 5-bromo-4-chloro-3-indolyl β-D-galactopyranoside (X-gal) or Bluo-Gal (beta-galactosidase), and the like.

The colored product can be detected by measurement of absorbance or transmittance of light by the colored product. In general, light is provided from a source that emits a spectrum of light in which at least one wavelength of light corresponds to the absorption spectrum of the colored product, typically in the wavelength range of about 250 nm to about 900 nm. Preferably, the color to be measured is in the visible range, or about 400 to about 800 nm. The spectrum of light emitted by the light source(s) accordingly is similar to the absorption spectrum of the colored product. Preferably, the emission spectrum from a light source will exactly overlap the absorption spectrum of the absorbing species, however, an exact overlap is not required. Monochromatic light sources and/or filters generally can be used to provide a means to match the characteristics of the absorption and the light source.

ELISA is the most common type of assay using an enzyme detection system, and includes the direct ELISA, sandwich ELISA, and competitive ELISA. In ELISA assays, a specific capture molecule that binds to the target analyte of interest is immobilized on a surface or other stationary solid phase, and is exposed in different steps to liquid reagents, usually with washing steps between exposures. In the final step, an enzyme substrate is added to produce an optical change, such as a color, that can be detected, usually by spectrophotometry.

In an exemplary direct ELISA, the target analyte sample to be assayed is added to the plastic reaction vessel (usually a microtiter plate) in a buffered solution where the target analyte adheres to the surface by charge interactions. A solution of non-reacting protein such as bovine serum albumin or milk protein then is incubated on the surface to “block” any of the plastic surface that is not coated with analyte, in order to reduce non-specific binding to the plate surface. A capture molecule, such as a monoclonal antibody or antibody substitute then is incubated in the reaction vessel and binds specifically to the bound analyte. This capture molecule bears an enzyme label. After washing unbound capture molecule away, the enzyme substrate is added and color develops as the enzyme catalyzes the reaction on the enzyme substrate. The more target analyte is in the sample, the more capture molecule is bound and the more enzyme is present, therefore the more substrate is converted to the colored, detectable product. The enzyme acts as a signal amplifier since one enzyme molecule can convert many molecules of substrate to the colored product. The intensity of color is read by spectrophotometry, with the possibility of a quantitative result by comparing to a series of controls of known concentration. An indirect ELISA is much the same as the direct ELISA described above, except that the capture molecule is not labeled. Capture molecule, such as an antibody, is detected using a secondary antibody that does bear an enzyme label and can bind specifically to the capture molecule, for example to the Fc region of a monoclonal antibody capture molecule.

In a sandwich ELISA, the surface of the reaction vessel is prepared with a known quantity of capture molecule. Any empty or non-specific sites on the vessel surface are blocked as described above. The sample containing target analyte is applied to the surface, where target analyte in the sample is captured. After washing to remove extraneous sample material and unbound analyte, a second capture molecule is added which also binds to the target analyte, forming a “sandwich.” The second capture molecule can comprise an enzyme label, or the second capture molecule alternatively can be detected by a third (labeled) molecule that binds to the second capture molecule, as described for the secondary antibody in the assay described above. The presence of enzyme is detected by adding substrate and reading a color change.

An exemplary competitive ELISA involves incubating unlabeled capture molecule in the presence of sample in solution. These bound complexes then are added to a reaction vessel having an analyte-coated surface. During incubation in the reaction vessel, the analyte coated to the surface competes for binding to the capture molecule. The more target analyte is present in the sample, the more capture molecule-analyte complexes are formed and the fewer unbound capture molecules are present to bind to the vessel surface. The vessel surface is washed to remove unbound material and a “secondary capture molecule” which is coupled to an enzyme and is specific to the capture molecule then is added. This labeled reagent binds to the surface where capture molecules have bound to the immobilized analyte. After washing again, substrate is added to detect any enzyme label bound to the surface. Alternative competitive ELISA tests use an enzyme-linked analyte molecule rather than an enzyme-linked secondary capture molecule. In this type of assay, the labeled analyte competes for binding to the capture molecule with the target analyte in the sample. The less target analyte in the sample, the more labeled capture molecule is retained in the reaction vessel and the more substrate conversion to colored product by the enzyme label.

Magnetic beads are made of iron oxide particles (about 5 to about 50 nm) encapsulated or glued together with polymer, and range in size from about 35 nm to about 4.5 μm. The particles exhibit superparamagnetism in the presence of an externally applied magnetic field. Magnetic particles or other carboxylated particles (gold or latex, for example) can be attached to a protein or peptide using the water-soluble carbodiimide EDC (EDAC), or by InnovaCoat GOLD™ or Innova LATEX™. Magnetic beads are non-reactive to and are not affected by typical assay reagents, are not affected to an appreciable degree by magnetic background in most instances, and are very stable. Magnetic beads can be used in various formats, including lateral flow assays (instead of gold labels), verticle flow assays, microfluidic and biochip applications, competitive, non-competitive and sandwich type assays and the like.

A magnetic immunoassay (MIA) uses magnetic beads as a label and optionally also as a separation method. These assays operate in the same general manner as ELISA. A magnetic bead is conjugated to one of the capture molecule or the analyte and the assay is run as describe above for ELISA. The assay can be performed “in solution” by applying a magnetic field to the reaction vessel to temporarily immobilize substances bound directly or indirectly to the magnetic beads against a vessel surface during washing or reagent removal steps as desired. At the end of the assay, the magnetic label in the vessel or in the wash solution can be measured by a magnetometer, which measures the magnetic field change induced by the beads or, when the particles to be measured are in solution, by light scattering techniques such as dynamic light scattering.

Colored dyes also can be used as labels and can be detected as described above for colored enzyme products with respect to ELISA. Colored particles of pigment also can be used, and are detected by colorimetric or light scattering methods. In principle, any colored particle can be used, however latex (blue or other colors) or nanometer sized particles of gold (red colour) are most commonly used. The gold particles are red in color due to localised surface plasmon resonance.

Metal particles (e.g. gold or silver nanoparticles) can be attached to many biomolecules, such as proteins, aptamers, and peptides (including antibodies and antibody substitutes), glycans, and nucleic acids (including aptamers, for example). Gold particles are commercially available (for example from Creative Biolabs™) in different sizes, which in solution produce different colors. Typically, these conjugations are achieved via primary amines on the biomolecule. Alternatively, gold particles specific to an analyte can be provided in the reaction chamber. Upon exposure to the fluid sample, the gold nanoparticles will bind to analyte contained in the sample creating a particle-biomolecular complex. These complexes can be detected using dynamic light scattering. See U.S. Pat. Nos. 8,883,094 and 9,005,994 and Liu et al. J. Am. Chem. Soc. 2008, 130, 2780-2782; for examples of detecting analytes using dynamic light scattering and metal particles.

A fluorophore is a fluorescent chemical compound that emits light of a specific wavelength upon excitation by light at a different, usually shorter, wavelength. These compounds are well known in the art for use as probes and indicators, and as labels or markers by covalent attachment to a reagent, such as a specific binding or affinity reagent in analytical methods. Fluorescent dyes are available commercially with fluorophores having excitation wavelengths including UV, visible and IR wavelengths from Bio-Rad™, Applied Biosytems, Perkin-Elmer and others.

Any of these compounds can be used with the invention for detection, including, but not limited to xanthene derivatives (such as fluorescein, fluorescein isothiocyanate, rhodamine, Oregon green, eosin, and Texas red), cyanine derivatives (such as cyanine, indocarbocyanine, oxacarbocyanine, thiacarbocyanine and merocyanine), squaraine derivatives (such as Seta, SeTau, and Square), naphthalene derivatives (such as dansyl and prodan derivatives), coumarin derivatives, oxadiazole derivatives (such as pyridyloxazole, nitrobenzoxadiazole, and benzoxadiazole), anthracene derivatives (such as anthraquinones, DRAQ5, DRAQ7 and CyTRAK Orange), pyrene derivatives (such as cascade blue), oxazine derivatives (such as Nile red, Nile blue, cresyl violet, oxazine 170), acridine derivatives (such as proflavin, acridine orange, acridine yellow), arylmethine derivatives (auramine, crystal violet, malachite green), tetrapyrrole derivatives (such as prophin, phthalocyanine, bilirubin), natural fluorophores (such as green fluorescent protein). Commercially available fluorophores also are available, including CF dye (Biotium™), DRAQ and CyTRAK probes (BioStatus™), BODIPY (Invitrogen™), Alexa Fluor (Invitrogen™), DyLight Fluor™ (Thermo Scientific™), Atto and Tracy (Sigma Aldrich™), FluoProbes (Interchim™), Abberior Dyes (Abberior™), DY and MegaStokes Dyes (Dyomics™), Sulfo Cy dyes (Cyandye™), HiLyte Fluor (Anaspec™), Seta, SeTau and Square Dyes (SETA BioMedicals™), Quasar and Cal Fluor dyes (Biosearch Technologies™), SureLight Dyes (Columbia Biosciences™), and APC, APCXL, RPE, and BPE (Phyco-Biotech™, Greensea™, Prozyme™, and Flogen™).

Fluorophores can be covalently linked to peptide functional groups such as amino, carboxyl, thiol, and azide groups. Such labels can be attached to protein, peptide and nucleic acid probes by, for example, the NHS (succinimidyl) ester method (many flurorescent dyes are available commercially with an activated NHS group), a heterobifunctional (two-tag) method (such as click chemistry using paired aromatic aldehyde and hydrazide tags, paired maleimide and thiol tags, and the like), a carbodiimide method (to link carboxyl and amine groups), isothiocyanate linking (commonly used with fluorescein), the Lightening-Link™ method, and the like.

Fluorescent detection methods are instruments that provide an excitation light source such as a laser, photodiode or lamp, filters to isolate specific wavelengths, and a detector that records the output, including but not limited to flow cytometry, fluorescence-activated cell sorting (FACS), fluorescent microscopy, spectrofluorometry, fluorometric microplate readers, fluorescent microarray readers and the like. Using digital methods including specialized software, the fluorescent signal can be detected quantitatively, to measure the amount of fluorophore.

An example of a fluorescence detection based assay is a direct method where sample containing target analyte is incubated with a fluorescent label-tagged capture antibody or aptamer, the bound capture molecule is separated from the unbound capture molecule by binding the bound target analyte to a second, unlabeled capture molecule which often is immobilized on a surface so that simple washing can remove bound from unbound label, and the fluorescent label remaining after separation is detected by spectrofluorometry. An exemplary indirect method uses a primary capture molecule immobilized on agarose beads, which is incubated with sample containing the target analyte, washed, and then incubated with a secondary molecule such as an antibody that bears a fluorescent label and specifically binds to the bound target analyte. The bound fluorescence then is detected and measured.

While some kind of label is generally employed in immunoassays, there are certain kinds of assays which do not rely on labels, but instead employ detection methods that don't require the modification or labeling the components of the assay. Surface plasmon resonance is an example of technique that can detect binding between an unlabeled antibody and antigens. Another demonstrated label-free immunoassay involves measuring the change in resistance on an electrode as antigen binds to it.

Chemiluminescent (emitting light as a result of a chemical reaction) and electrochemiluminescent (emitting light in response to electric current) labeling methods also can be used with the assay formats discussed herein. These labels perform similarly to fluorescent labels and are used in a similar manner, and are detected using specialized instruments.

Radiolabeling can be achieved by iodination of certain amino acids (usually tyrosines) in proteins and peptides, by introducing phenolic sites for iodination using the Bolton-Hunter reagents, SHPP and sulfo-SHPP, by adding a crosslinker containing one or more tyrosyl groups, or by substituting an amino acid residue in the sequence with a tyrosine. Radioactive ¹²⁵I or ¹³¹I can be incorporated by enzymatic or chemical oxidation, as is known in the art. Other radiolabels also can be used, such as ³H, ¹³C, ¹¹C, ¹³N, ¹⁵O, ¹⁸F, ³²P, ³⁵S, ^{99m}Tc, and the like. Radiolabeled amino acids, nucleotides, and the like, for incorporation into a capture molecule or analyte are available commercially. Assays can be performed according to standard methodologies as described herein, usually as a radioimmunoassay (RIA) or immunoradiographic assay (IRA).

Traditional RIAs are performed by radioactively labeling the analyte to be detected and mixing it with a known amount of capture molecule specific for the analyte to form complexes. These complexes then are incubated with the sample containing target analyte. The unlabeled target analyte in the sample competes for binding to the capture molecule, displacing the label from the complex. The bound analyte and unbound analyte are separated, and the radiolabel in one or both of these fractions is measured using a gamma counter or a scintillation counter. These assays also can be performed using a sandwich format as described above for ELISA. Radioactive isotopes can be incorporated into most binding assay reagents to produce a radioimmunoassay (RIA). Radioactivity emitted by bound antibody-antigen complexes or unbound reagents can be easily detected using conventional methods. Classically, RIAs were some of the earliest immunoassays developed, but have fallen out of favor largely due to the difficulty and potential dangers presented by working with radioactivity.

Proximity ligation assays (PLA) allow direct detection of proteins and protein interactions with high sensitivity. These assays involve two primary antibodies, each of which recognize and bind to the same target analyte, and which bear short, unique sequences of DNA. When the DNA strands on the two antibodies are in close proximity (about 30-40 nm), by binding two different epitopes on the same target analyte, the DNA strands can interact. Two additional DNA molecules, connector oligonucleotides, are introduced and are ligated enzymatically to the DNA strands on the primary antibodies, leading to the formation of a circular, single-stranded DNA molecule. In this circle, one of the DNA strands serves as a primer for rolling circle amplification (RCA). Therefore, when a DNA polymerase is added, a long DNA product forms and remains attached to one of the PLA probes. The several hundred-fold replication of the same sequence in one long molecule enable hybridization of multiple detection oligonucleotides (usually labeled with fluorescence) and detection of the product by visualized under a microscope and quantitation. This technique is described for in situ visualization of protein complexes in Söderberg et al., “Direct observation of individual endogenous protein complexes in situ by proximity ligation. Nat. Methods 3(12):995-1000, 2006. The principle is the same for the indirect form of PLA, however in this method the unmodified primary antibodies are raised in different species of animals and detected with two secondary antibodies that are equipped with the DNA strands.

### I. Instrumentation

In a specific embodiment, analyte detection devices include a light source (either for illuminating the test vessel or for providing light or exciting the fluorescent reagents) and/or sensors for detecting the light emitted from the reagents, and/or a processor for calculating and presenting the results of the assay. These elements can be associated with the housing of the analyte detection device or in a separate unit.

A colored product of an analyte-detecting assay of the present invention is typically detected by measurement of absorbance of light by the colored product. Light will be directed to the colored product in a reaction site from a source that emits a spectrum of light in which at least one wavelength of light corresponds to the absorption spectrum of the colored product. The spectrum of the light emitted by a source accordingly will be similar to the spectrum of the absorbing species in the colored product of the analyte-detecting reaction. Preferably, the emission spectrum from the light source will overlap the absorption spectrum of the absorbing species, preferably by at least about 50%, 60%, 70%, 80%, 90% or 95%. However, the present invention does not require an exact overlap between the light source emission spectrum and the absorption spectrum of the colored product, as described in the examples provided herein. Use of monochromatic light sources and/or filters can generally provide a means to match the characteristics of the absorption and the light source. The colored products detected by the subject system typically have an absorption range of about 250 nm to about 900 nm. Preferably, the color to be measured is generally in a visible range of about 400 to about 800 nm.

Alternatively, the LED and/or sensors can be integrated into the disposable cassette. In such alternative embodiment, the analyte detection device can comprise a display, (rechargeable) battery, memory, bar code reader for patient ID, data port or wireless technology for electronic record keeping.

The absorbance of the colored product can be readily detected and in a range that is preferably stoichiometrically or linearly corresponds to the amount of analyte present. According to Beer's law, absorbance=concentration X.extinction coefficient.X optical path length. Chromophores in the visible wavelength range and typically used in clinical chemistry have extinction coefficients in the range of about 10³-10⁵ L/(mole×cm). By way of example, a concentration of 1.5 mM analyte, diluted by 1:30 fold, may give an absorbance of 0.25 (44% transmission) when measured at the maximum absorbance (at λ max of 500 nm, the extinction coefficient=50,000 L/(mole×cm) with a path length of 0.1 cm (typical of single use cartridges). This absorbance is readily measurable by simple transmission optical systems.

A variety of light sources may be utilized for the present invention depending on the particular type of application and absorbance spectrum requirements for a given analyte of interest. An example of an appropriate light source includes, but is not limited to, an incandescent bulb, a light emitting diode, luminescent paint, and a laser. Preferably, the light source is an economical, low intensity light source well suited for point-of-care testing. When coupled with a photomultiplier tube detector, the number of photons generated by the light source need only be a few thousand over a measurement interval, which can range from a few milliseconds to a several minutes.

One type of light source applicable for the present invention is luminescent paint. Such paint is generally formulated using very tiny quantities of a long-lived radioisotope together with a material that glows or scintillates non-destructively when irradiated. The paint can be appropriately colored by addition of dyes. The paint will generally be coated on the non-transparent walls of a reaction site where analyte assay chemistry generates a colored product. Light emitted from the paint can be detected through a transparent surface of the reaction site to allow measurement of absorbance due to a colored product. The spectrum of the light emitted will generally be a function of the scintillant material and the absorbance characteristics of the chemistry used in forming a colored product.

Another applicable light source for the present invention is a Light Emitting Diode (LED). A LED can provide colored light at moderate intensity. The spectrum of the emitted light can be selected over the visible range. A LED typically has a more narrow range of emission wavelengths of about 30 nm. Thus, use of a LED as a light source will depend on the absorbance spectrum of an absorbing species used in the detection of a particular analyte.

Detection and measurement of colored products generated due to the presence of a given analyte can be made directly from a reaction site or alternatively from a detection site to which the colored product is transported. Preferably, detection will be made from a reaction site. Unless specified otherwise, the term “reaction site” as used herein will refer to both the site at which a reaction occurs and at which the colored product of the reaction is detected. The reaction site will typically be a well that is cylindrical in shape having a defined length between two opposed flat surfaces for determination of absorbance. For example, the point-of-care fluidic devices of the present invention may have a reaction site that is 0.1-1 cm in length. At least one or both of the flat surfaces of the reaction site will be transparent to allow detection of the colored product with standard transmission optics. The non-transparent surfaces of the reaction site may be made of opaque, white light scattering material. The detector of light transmitted from a light source through a reaction site will be capable of detecting absorbance of light by the colored product in the reaction site. Examples of suitable detectors include, but are not limited to, a photomultiplier tube, a photodiode or an avalanche photodiode. In a system of the present invention, the position of the light detector in the system relative to the fluidic device will depend on factors such as the type of light source used and the relative position of the light source to the fluidic device. In the case where the light source is a luminescent paint contained within a reaction site of the device, the detector will be positioned to detect light emitted from a transparent surface of the reaction site.

The analyte detection device, in addition to having a reaction chamber with reagents that interact with an ocular analyte, also can include a light positioned to illuminate the assay reaction chamber and a sensor to detect light reflected from the reaction chamber. The analyte detection device also can include a processor, with optional memory component, to process the signal provided by the sensor to determine an amount of signal, compare the signal to stored values and/or provide qualitative or quantitative readout, which can be provided on a display component associated therewith.

n a typical embodiment, the analyte detection device includes a housing having an inlet connected (e.g. attached or integral) to the aspiration conduit and/or an outlet connected to the aspirator. The housing may also be transparent or include a window allowing visibility into the reaction chamber. The analyte detection device may further include a light for illuminating the reaction chamber. The term light as used herein is intended to include a device that can generate of electromagnetic radiation. The light may be implemented in a fashion such that the label of the labeled conjugate reagent is detectable in the reaction chamber.

In an alternative embodiment, the analyte detection device also includes a sensor that is implemented to sense the presence of label on the reaction chamber. The sensor typically is one that can detect electromagnetic radiation. For example, the sensor may detect a radiation from a radioactive isotope label, fluorescence from a fluorescent label, color amount/intensity from a color signal (e.g. such as red color produced from gold label or horseradish peroxidase), etc.

In the situation where the light source is external to a fluidic device, a detector could be positioned either on the same side or an opposite side of the fluidic device relative to the light source. A reaction site can be configured with a single transparent surface, through which light is both directed to the reaction and detected from the reaction. In this scenario, a detector is positioned on the same side of the fluidic device as the light source, with the detector shielded such that the only light detected is that from the reaction site of the fluidic device. Alternatively, a reaction site can be configured with two flat, opposed transparent surfaces such that the reaction site is effectively an optical cuvette. In this configuration, the light source would emit light to one side of the reaction site in the fluidic device and the detector would detect the light transmitted through the colored product to the opposite side of the reaction site in the fluidic device.

### J. Other Components

Other reagents useful in embodiments of the present invention include without limitation, salts, wash buffers, enzyme substrates, conjugates, enzyme-labeled conjugates, DNA amplifiers, diluents, detergents, surfactants, thickeners, chaotropes, preservatives, pH adjusters, polymers, chelating agents, albumin-binding reagents, enzyme inhibitors, enzymes, anticoagulants, red-cell agglutinating agents, antibodies, or other materials necessary to run an assay in a fluidic device. In general, reagents especially those that are relatively unstable when mixed with liquid are confined in a defined region (e.g. a reagent chamber) within the subject fluidic device. The containment of reagents can be effected by valves that are normally closed and designed for one-time opening, preferably in a unidirectional manner or by containment in a container that can be pierced or broken to release the contents. In some embodiments the reagents are initially stored dry and dissolved in a solution to initiate the assay. In some embodiments, a reactant site, reactant chamber or reagent chamber contains approximately about 50 μL to about 1 mL of fluid. In some other embodiments, the chamber contains about 100 μL to about 1 mL of fluid. In some embodiments, the chamber contains about 100 μL of fluid. The volume of liquid in a reactant or reagent chamber may vary depending on the type of assay being run or the sample of bodily fluid provided. Solvents and diluents can be aqueous, such as buffered saline, or can contain organic solvents as suitable for the reaction used in the assay. Organic solvents such as ethanol, methanol, DMSO, THF, and the like are contemplated.

K. Methods of Use with Treatment

According to another embodiment, provided is a method of determining efficacy of an agent administered to an eye. The method involves obtaining a vitreous humor sample from a subject; subjecting the vitreous humor sample to a reaction chamber comprising reagents for performing a heterogenous or homogenous assay as described herein; detecting an amount of the at least one analyte in the vitreous humor sample; and correlating the amount of the at least one analyte with a predetermined level or range, wherein if the at least one analyte is at or within the predetermined level or range, respectively, the agent is determined as effective. The method may further involve administering a first dose of an agent to the eye of the subject and optionally administering a second dose if the amount of the least one analyte is outside the predetermined level or range. In a specific embodiment, the dosage amount of the second dose is adjusted based on the amount of at least one analyte

According to another embodiment, a method of determining efficacy of an agent administered to an eye is provided. The method involves obtaining a vitreous humor sample from a subject; subjecting the vitreous humor sample to reaction chamber comprising reagents to detect at least one analyte of interest via a homogenous assay as described herein; detecting an amount of the at least one analyte in the vitreous sample; and correlating the amount of the at least one analyte with a predetermined level or range, wherein if the at least one analyte is at or within the predetermined level or range, respectively, the agent is determined as effective.

### 5. Examples

This invention is not limited to the particular processes, compositions, or methodologies described, as these may vary. The examples below therefore are intended to be exemplary and not limiting.

### Illustrated Assay System

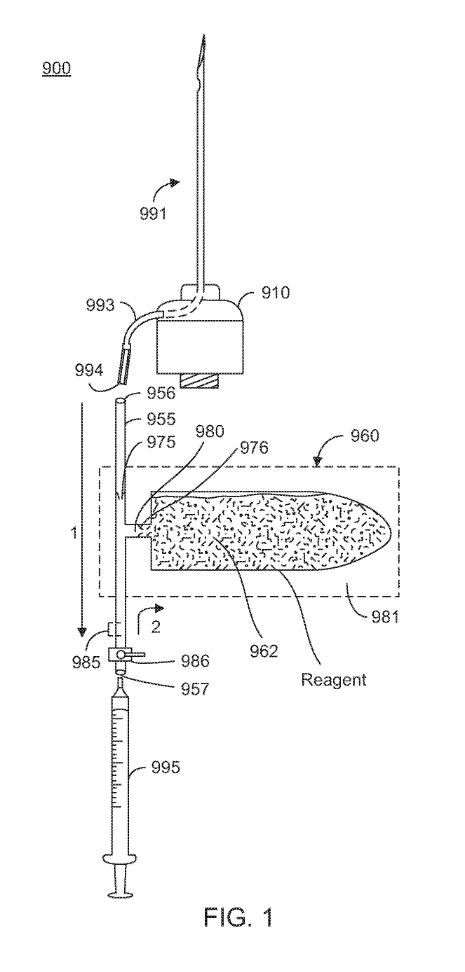

Referring to FIG. 1, shown is a vitreous sample acquisition and assay system 900 particularly configured for a liquid-based analyte detection. The system 900 includes a sample acquisition device 991 that includes a probe 910 having a cannulated needle body 912 and aspiration inlet 914 and an aspiration channel 916 in fluid communication with the aspiration inlet 914. The sample acquisition device 991 can be associated with a vitrectomy device (not shown). The aspiration channel 916 is in fluid communication with an aspiration conduit 993 that has an aspiration conduit proximal portal 994. The system 900 also includes a sample conduit 955 having a sample conduit distal portal 956 that associates with the aspiration conduit proximal portal 994.

Associated with the sample conduit 955 is an analyte detection device 960. The analyte detection device 960 includes a housing 981 that contains a reaction chamber 962 into which analyte reagents are located. The reaction chamber 962 is in fluid communication with a shunt 980 that is in fluid communication with the sample conduit 955. The sample conduit 955 includes a sample conduit valve 975 that is positioned distally to the shunt 980. The sample conduit valve 975 is a one-way valve that permits flow from a distal to a proximal direction. The shunt 980 also includes a shunt valve 976 between the sample conduit 955 and the reaction chamber 962. The shunt valve 976 is a one way valve that permits flow from the sample conduit 955 to the reaction chamber 962.

The system 900 also includes as aspirator (shown as syringe) 995 that associates with the sample conduit proximal portal 957. Provided on the sample conduit 955 are fill indicia 985.

Use of the system 900 involves step 1: the acquisition of a vitreous sample. During step 1, while the aspiration conduit 993 and sample conduit 955 are connected, a vacuum is applied by the aspirator 995 and sample is drawn into the sample conduit 955 until it reaches the fill indicia 985. During aspiration, the sample conduit valve 975 opens to allow sample to pass and shunt valve 976 is shut. Once sample reaches the fill indicia 985, step 2 is initiated: directing sample into the reaction chamber 962. During step 2, the aspirator 995 applies pressure to sample conduit 955. As pressure is applied to sample conduit 955, sample conduit valve 975 shuts and shunt valve 976 opens to allow sample to pass through the shunt 980 and into the reaction chamber 962.

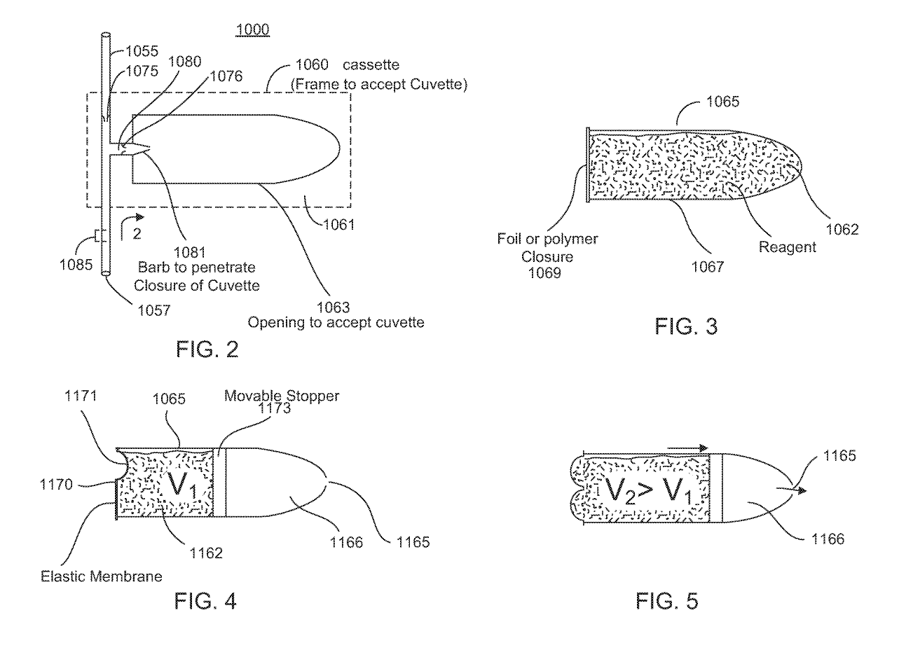

FIG. 2 shows an alternative embodiment 1000 that is similar to embodiment 900. However, in embodiment 1000, the analyte detection device 1060 includes a housing 1061 that has a receptacle 1063 for receiving a cartridge 1065. The cartridge 1065 has a cartridge housing 1067 that defines a reaction chamber 1062 (see FIG. 3). As shown in FIG. 3, the cartridge 1065 may include a pierce-able closure 1069. The shunt 1080 shown in FIG. 2 includes a sharp end 1081 that can puncture the closure 1069 upon association of the cartridge 1065 in the receptacle 1063, thereby establishing fluid communication between the shunt 1080 and the reaction chamber 1062.

FIG. 4 shows an alternative cartridge 1065 that allows for compensation of the delivered sample volume. One compensation approach relates to use of an expandable closure 1170, such as an elastic membrane. Another compensation approach relates to the implementation of a feature 1171, such as a bubble, on the closure 1170. The feature may shift to allow for more volume, for example, the bubble inverts to provide more space in the reaction chamber 1162. Another compensation approach involves the implementation of a movable stopper 1173 positioned within the reaction chamber 1162. When implementing the movable stopper 1173, the cartridge 1065 is equipped with a vent 1165 and a venting region 1166 that is positioned between the movable stopper 1173 and the vent 1165. To compensate for increased sample volume, the movable stopper 1173 may shift or move toward the vent 1165 (as shown in FIG. 5), which causes a decrease of the venting region 1166 and expulsion of air out the vent 1165. It is noted that one, two or all three of the foregoing approaches may be implemented to compensate for volume in the reaction chamber 1162.

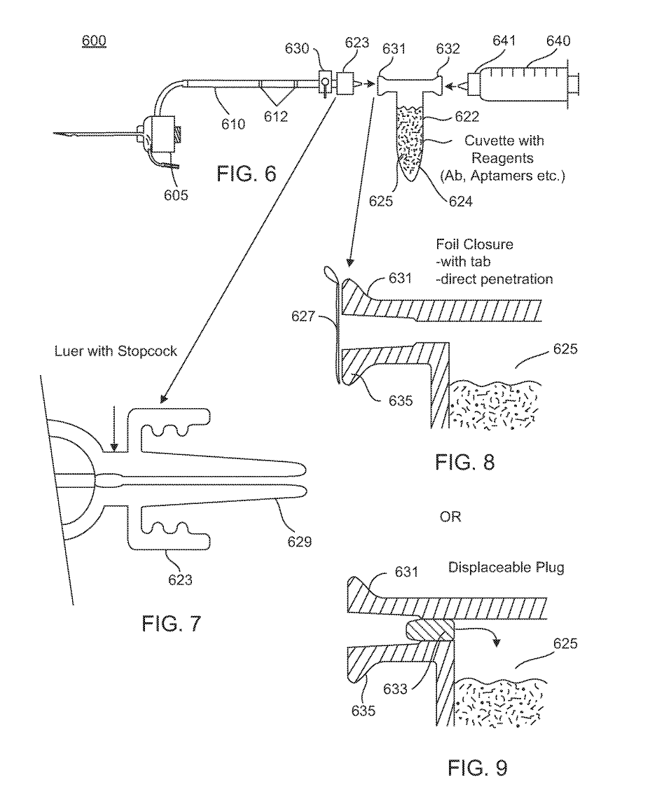

Turning to FIG. 6, shown is an alternative system embodiment 600 that provides for sample acquisition and analyte detection. The system 600 involves a sampling line 610 having fill indicia 612. The sampling line 610 is associated with a sample acquisition device 605 at the distal end and an analyte detection device 622 at the proximal end. The analyte detection device 622 includes a container 624 that defines a reaction chamber 625 therein. The sample acquisition device 605 is typically a vitrectomy probe such as that described in FIG. 1 (e.g. 991) which can be associated with a vitrectomy device (not shown). The sampling line 610 may or may not be removably connectable to the sample acquisition device 605. The sampling line 610 may have a connector 623 at the proximal end (see FIG. 7 for close up of connector 623 and tip 629). Distal to the connector 623 is a valve 630 (e.g. manual valve such as a stopcock).

The analyte detection device 622 has an inlet 631 and outlet 632. The proximal end of the sampling line 610 associates with the inlet 631 and an aspirator 640 associates with the outlet 632. As shown, the aspirator 640 is a syringe that comprises a connector 641 that engages the outlet 632. The inlet 631 comprises a connecting portion 635 that interacts with the connector 623. In one example shown in FIG. 8, the inlet 631 has a closure 627 that may be peeled off before use or punctured by the tip 629 of the connector 623 (see FIG. 7) upon association between the connector 623 and the inlet 631 thereby allowing fluid communication with the reaction chamber 625. Alternatively, as shown in FIG. 9, the inlet 631 may include a displaceable plug 633. The plug 633 has a diameter greater than the inner diameter of the tip 629 of the connector 623. Upon association of the connector 623 with the inlet 631, the tip 629 pushes the plug 633 into the reaction chamber 625. It is noted that the association of the aspirator 640 and the outlet 632 may be one of the configurations described above for the association between the connector 623 and the inlet 631.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the preceding definitions are provided.

While a number of embodiments of the present invention have been shown and described herein in the present context, such embodiments are provided by way of example only, and not of limitation. Numerous variations, changes and substitutions will occur to those of skill in the art without materially departing from the invention herein. For example, the present invention need not be limited to best mode disclosed herein, since other applications can equally benefit from the teachings of the present invention. Also, in the claims, any means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

While one or more embodiments of the present invention have been shown and described herein, such embodiments are provided by way of example only. Variations, changes and substitutions may be made without departing from the invention herein. Accordingly, it is intended that the invention be limited only by the spirit and scope of the appended claims. The teachings of all references cited herein are incorporated in their entirety to the extent not inconsistent with the teachings herein.

### 6. References

All publications, including patents and patent applications referenced herein are hereby incorporated by reference in their entirety.

What is claimed is:

1. A system for detecting a target analyte in a sample, comprising:
a) a sample acquisition device comprising a cannulated needle with an aspiration inlet, and an aspiration channel in fluid communication with the aspiration inlet;
b) an aspiration conduit in fluid communication with the aspiration channel, wherein the aspiration conduit comprises an aspiration conduit proximal portal;
c) a sample conduit that comprises a sample conduit distal portal that associates with the aspiration conduit proximal portal; and
d) an analyte detection assay device comprising a housing that comprises a reaction chamber that is in fluid communication with the sample conduit and which contains reagents that specifically interact with the analyte.

2. The system of claim 1, wherein the reaction chamber is in fluid communication with a shunt that is in fluid communication with the sample conduit.

3. The system of claim 2, wherein the sample conduit comprises a sample conduit valve that is positioned distally to the shunt.

4. The system of claim 3, wherein the sample conduit valve is a one-way valve that permits flow of liquid from a distal to a proximal direction.

5. The system of claim 2, wherein the shunt comprises a shunt valve positioned between the sample conduit and the reaction chamber.

6. The system of claim 5, wherein the shunt valve is a one way valve that permits flow from the sample conduit to the reaction chamber.

7. The system of claim 1, further comprising an aspirator that is in fluid communication with the sample conduit.

8. (canceled)

9. The system of claim 7, wherein the sample conduit comprises one or more fill indicia, positioned between the shunt and the aspirator.

10. (canceled)

11. The system of claim 1, wherein the reaction chamber comprises a removable cartridge that associates with the housing at receptacle defined therein.

12. The system of claim 11, wherein the cartridge comprises a closure that is punctured by the shunt upon placement into the receptacle.

13. (canceled)

14. The system of claim 12, wherein the closure comprises an elastic membrane that stretches to accommodate excess sample volume.

15. (canceled)

16. (canceled)

17. The system of claim 1, wherein the reaction chamber comprises a movable stopper and a vent to accommodate excess sample volume.

18. A system for detecting a target analyte in a sample, comprising:
a) a sample acquisition device comprising a cannulated needle with an aspiration inlet, and an aspiration channel in fluid communication with the aspiration inlet;
b) a sampling line comprising a distal end and a proximal end, wherein the sampling line is in fluid communication with the aspiration channel at the distal end; and
c) an analyte detection assay device comprising a housing that comprises a reaction chamber that is in fluid communication with the proximal end of the sampling line and which contains reagents that specifically interact with the analyte, wherein the sampling line comprises one or more fill indicia positioned between said distal and proximal ends.

19. The system of claim 18, wherein the sampling line comprises a connector at the proximal end that interacts with an inlet of the housing.

20. (canceled)

21. The system of claim 18, further comprising an aspirator that is in fluid communication with the housing.

22. (canceled)

23. The system of claim 19, wherein the inlet comprises (i) a removable closure or (ii) a puncturable closure that is punctured by the connector upon connection to the inlet.

24. (canceled)

25. The system of claim 19, wherein the inlet comprises a displaceable plug that pushes into the housing upon connection of the connector to the inlet.

26. (canceled)

27. The system of claim 18, wherein the sampling line comprises a valve distally positioned relative to the housing.

28. (canceled)

29. The system of claim 27, wherein the one or more fill indicia are positioned distally to the valve.

30. (canceled)

31. (canceled)

32. The system of claim 1, wherein the at least one analyte comprises an ocular analyte.

33. The system of claim 32, wherein the ocular analyte comprises an angiogenic ocular analyte or an inflammatory ocular analyte.

34. The system of claim 33, wherein the angiogenic ocular analyte comprises VEGF and the inflammatory ocular analyte comprises IL-6.

35. (canceled)

36. A method of detecting at least one analyte in a sample, the method comprising:
obtaining a system according to claim 1;
acquiring a vitreous humor or aqueous humor sample using the sample acquisition device of system; and
detecting the at least one analyte in the vitreous humor or aqueous humor sample using the analyte detection device of the system, wherein the reaction chamber comprises one or more reagents for detecting the at least one analyte via a homogenous assay.

37. (canceled)

38. (canceled)

39. A method of detecting at least one analyte in a sample, the method comprising:
obtaining a system according to claim 18;
acquiring a vitreous humor or aqueous humor sample using the sample acquisition device of system; and
detecting the at least one analyte in the vitreous humor or aqueous humor sample using the analyte detection device of the system, wherein the reaction chamber comprises one or more reagents for detecting the at least one analyte via a homogenous assay.
